(19) United States
(12) Patent Application Publication
(10) Pub. No.: US 2011/0129414 A1
(43) Pub. Date: Jun. 2, 2011

(54) Single Chain Fragment of Monoclonal Antibody 9B9 and Uses Thereof

(76) Inventors: Irina V. Balyasnikova, Chicago, IL (US); Sergei M. Danilov, Oak Park, IL (US)

(21) Appl. No.: 12/093,770

(22) PCT Filed: Nov. 15, 2006
(86) PCT No.: PCT/US06/44382

§ 371 Date: Apr. 21, 2010

Related U.S. Application Data
(60) Provisional application No. 60/736,897, filed on Nov. 15, 2005.
(60) Provisional application No. 60/802,468, filed on May 22, 2006.

(51) Int. Cl. A61K 51/10; C07K 16/40; A61K 39/395; C07H 21/04; C12N 15/63; C12N 5/10; C12N 1/21; C12P 21/02; A61K 35/00

(52) U.S. Cl. 424/1.49; 530/389.8; 424/178.1; 536/23.53; 435/320.1; 435/419; 435/325; 435/348; 435/252.3; 435/69.6; 424/93.2; 530/391.3; 530/391.7

(57) ABSTRACT

Anti-ACE single chain fragment antibodies are disclosed. The present invention relates to using these antibodies, and polymers thereof, in methods for detecting, diagnosing, prognosing, preventing, or treating diseases associated with ACE expressing tissue.

### RELATED APPLICATIONS

This application claims the benefit of U.S. Patent Application No. 60/736,897, filed on Nov. 15, 2005 and U.S. Patent Application No. 60/802,468, filed on May 22, 2006. Both applications are hereby incorporated by reference in their entirety.

### TECHNICAL FIELD

The present invention generally relates to the field of immunology. More specifically, the present invention relates to the cloning and expression of single chain fragments of the monoclonal antibody 9B9 and uses thereof.

### BACKGROUND OF THE INVENTION

The lung endothelium is susceptible to oxidative injury and this has pathological significance for a number of diseases. Angiotensin-converting enzyme (ACE) is an important regulator of blood pressure. In addition to being expressed in epithelial cells, ACE is also expressed on the luminal surface of endothelial cells. However, endothelial ACE is expressed in a vessel- and species-specific manner. Several studies have demonstrated that ACE is a suitable target for the specific delivery of drugs to the lung vasculature using anti-ACE monoclonal antibodies (mAbs) as carriers. After systemic injection, anti-ACE mAb 9B9 selectively accumulates in the lungs of several mammals, including humans. Conjugation of anti-ACE mAB 9B9 to plasminogen activators, catalase or superoxide dismutase results in the specific targeting of these drugs and prolonged association with the pulmonary vasculature. The therapeutic relevance of this approach is supported by the observation that conjugates of catalase with anti-ACE mAb 9B9 diminished the damage of the endothelium by hydrogen peroxide in isolated perfused lung. This anti-ACE antibody was successfully used to redirect viral vector to the pulmonary circulation and to increase the selectivity and efficacy of lung transgene expression. Thus, using a bi-specific conjugate antibody for re-direction of adenoviruses to ACE, 20-fold enhancement of pulmonary gene delivery and expression in vivo, along with significantly reduced (5-8 fold) transgene expression in non-targeted organs was achieved. See Reynolds et al., Nature Biotech 19:838-842, 2001. Moreover, the combination of transductional retargeting adenoviruses (via ACE) and transcriptional retargeting (with the use of an endothelial specific promoter for vascular endothelial growth factor receptor 1, flt-1) resulted in a remarkable, highly synergistic improvement in selectivity of transgene expression in the lung compared to the usual site of vector sequestration, the liver. An improvement in relative selectivity of 300,000-fold for lung:liver expression, and 6000-fold for lung:spleen expression when compared to non-targeted vector has been observed (Reynolds P. M. et al., 2001). Thus, antibody-directed lung-selective gene delivery via ACE shows tremendous potential (Pinckard R. N. and Weir, D. M., 1978; Pimm M. V., 1995). As a confirmation of this potential, systemic administration of adenoviruses encoding eNOS, chemically conjugated with anti-ACE mAb 9B9, enhanced eNOS expression in the rat lung and attenuated the systemic hypertension in SHR-SP rats (Miller W. H. et al., 2005).

To date, anti-ACE mAb 9B9 is the only mAb to react with rat ACE and accumulate in the rat lung after systemic injection (Danilov et al., 1991; Danilov et al., 1994), and humans (Muzykantov V. R. and Danilov S. M., 1995). However, the clinical utility of mAb 9B9 can be limited due to its murine source and presence of Fc fragment. The utility of Fab and scFv fragments instead of whole IgG should be advantageous due to the lack of Fc fragments, which further reduce their possible side effects (Woof, J. M. and Burton D. R., 2004). Such scFv or Fab antibody fragments to ACE could be valuable reagents in the treatment of pulmonary diseases by placing them on the surface of vehicles (liposomes, polymers or viruses) carrying genes encoding a variety of potential genes of interest or by preparing genetic fusions encoding different active substances. ScFv antibody fragments could be obtained directly from antibody producing hybridoma cell lines by genetic engineering or from numerous existing human and animal phage display libraries.

Previous efforts to achieve these desired improvements in tissue-specific targeted therapy and treatment have centered on the use of monoclonal antibodies, antibody fragments and other proteins or polypeptides (i.e., molecular weight over 10,000 D) that bind to, for example, tumor cell surface receptors. The specificity of these pharmaceuticals is frequently very high, but they suffer from several disadvantages. First, because of their high molecular weight and their use in many radiopharmaceutical diagnostics, they are generally cleared from the blood stream very slowly, resulting in a prolonged blood background in the images. Also, due to their molecular weight they do not extravasate readily at the targeted site and then only slowly diffuse through the extravascular space to the targeted cell surface. This results in a very limited amount of the radiopharmaceutical reaching the receptors and thus very low signal intensity in imaging and insufficient cytotoxic effect for treatment. These pharmacokinetic properties also result in low tumor-to-background ratios for most intact mAbs and have limited their use as diagnostic imaging agents. In contrast, antibody fragments and appropriately engineered antibody species generally exhibit faster blood clearance, with lower liver and splenic uptake, while maintaining high antigen specificity and affinity. Furthermore, with the relatively recent development and use of single chain fragment antibodies (scFvs), biodistribution is accelerated, with lower retention and faster blood clearance. These fragments elicit little or no immune response after administration to patients because they last only a short time in circulation. Because of their smaller size, as compared to a full length antibody, scFvs diffuse through the patient's vascular compartment. These characteristics make scFvs ideal carriers of therapeutic as compared to antibodies.

Many contributions to antibody engineering were made years in advance of the modern imaging technology and therapy needed to best realize their full potential as imaging agents. Furthermore, recombinant DNA techniques have been used to generate chimeric antibodies having murine variable regions and human constant regions. Such chimeras generate a substantially less immunogenic reaction in humans than murine mAbs.

A need remains for harnessing these technologies to better direct therapeutics and imaging agents to specific tissues. The present invention seeks to ameliorate the foregoing disadvantages of the prior art by providing compounds which exhibit stable complex formation with the selected therapeutic, drug, or radionuclide to be delivered; are of sufficient limiting size to allow for an optimal serum half-life, penetrate the tissue, diseased tissue, or tumor, and optimally diffuse from the vasculature.

Described herein are methods for cloning and preparing a functional scFv fragment from an existing hybridoma cell line producing mAb 9B9, and the characterization of its binding with rat and human ACE in vitro and in vivo. mAb 9B9 can be purchased from Chemicon, International, Temecula, Calif.

### SUMMARY OF THE INVENTION

The present invention provides an isolated nucleic acid molecule (SEQ ID NO:1) which encodes the scFv antibody (See FIG. 1, “scFv9B9”, for example). Furthermore, the present invention relates to vector constructs comprising SEQ ID NO:1. The vectors of the present invention can be, for example, plasmids, cosmids, phagemids, YACs, or BACs.

The present invention provides an isolated nucleic acid molecule (SEQ ID NO:19) which encodes the scFv antibody (See FIG. 14, “scFv9B9(N68Q)”, for example). Furthermore, the present invention relates to vector constructs comprising SEQ ID NO:19. The vectors of the present invention can be, for example, plasmids, cosmids, phagemids, YACs, or BACs.

The present invention also relates to the amino acid sequence encoded by the nucleotide sequence SEQ ID NO:19 (SEQ ID NO:18), or to the amino acid sequence encoded by the nucleotide sequence SEQ ID NO:1 (SEQ ID NO:2). In particular, the present invention relates to scFv antibodies which specifically bind to angiotensin converting enzyme (“ACE”). These antibodies comprise, or alternatively consist of, the amino acid sequence of SEQ ID NO:18. In another embodiment, these antibodies comprise, or alternatively consist of, the amino acid sequence of SEQ ID NO:2.

An object of the present invention is to provide a conjugated antibody complex comprising one or more selected drugs conjugated to one or more anti-ACE scFv 9B9 antibodies capable of delivering the selected drug to an ACE-expressing tissue, Examples of ACE-expressing tissue include, but are not limited to, endothelial cells epithelial cell of epididymis, small intesting and probimal tubules of the kidney, alveolar macrophages and neuronal cells of basal ganglia. However, anti-ACE monoclonal antibodies do selectively accumulate in the lung endothelium. See Danilov et al., A.J. Physiol Lung Physiol. 2001. In a preferred embodiment, the herein disclosed scFv antibodies are used in therapies which specifically target the lung endothelium. The herein disclosed antibodies may take the form of a polymeric antibody, wherein two or more scFv antibodies are engineered into multimers. Furthermore, the herein described antibodies may be used in the manufacture of various medicaments for the directed treatment of ACE-expressing tissues.

In preferred embodiments, the ACE binding antibodies of the invention bind to the ACE enzyme in its native conformation. In another embodiment, the ACE binding antibodies of the present invention bind with high affinity to ACE like peptides or polypeptides that exhibit a native conformation. In yet other embodiments, the present invention provides ACE scFv antibodies which are, or can be, attached, coupled, linked or adhered to a matrix or resin or solid support. Techniques for attaching, linking or adhering polypeptides to matrices, resins and solid supports are well known in the art. Suitable matrices, resins or solid supports for these materials may be any composition known in the art to which an ACE binding polypeptide of the invention could be attached, coupled, linked, or adhered, including but not limited to, a chromatographic resin or matrix, the wall or floor of a well in a plastic microtiter dish, such as used in ELISA assays, or a silica based biochip. Materials useful as solid supports on which to immobilize binding polypeptides of the invention include, but are not limited to, polyacrylamide, agarose, silica, nitrocellulose, paper, plastic, nylon, metal, and combinations thereof. An ACE binding antibody of the present invention may be immobilized on a matrix, resin or solid support material by a non-covalent association or by covalent bonding, using techniques known in the art.

The present invention also relates to recombinant vectors, which include the isolated nucleic acid molecules encoding the ACE binding antibodies of the present invention (as well as variants thereof), and to host cells containing the recombinant vectors, as well as to methods of making such vectors and host cells. The invention further provides for the use of such recombinant vectors in the production of ACE binding scFv antibodies by recombinant techniques.

The ACE binding scFv antibodies of the present invention, nucleic acids, transformed host cells, and genetically engineered viruses and phage of the invention (e.g., recombinant phage), have uses that include, but are not limited to, the detection, isolation, and purification of ACE; the treatment and therapy of diseased tissue associated with ACE; and the diagnosis of diseases tissue associated with ACE. For example, the ACE binding scFv antibodies of the present invention may be used for the manufacture of a medicament for the treatment and therapy of a diseased tissue associated with ACE.

The present invention also encompasses methods and compositions for detecting, treating, diagnosing, prognosing, and/or monitoring diseases or disorders associated with aberrant ACE expression; detecting, treating, diagnosing, prognosing, and/or monitoring diseases or disorders associated with a tissue that expresses ACE. Diseases and disorders which can be detected, treated, diagnosed, prognosed, and/or monitored with the ACE binding scFv antibodies include those of an animal, preferably a mammal, and most preferably a human. Diseases and disorders which can be detected, diagnosed, prognosed and/or monitored with the ACE binding polypeptides of the invention include, but are not limited to, cardiovascular disorders (e.g., hypertension, chronic heart failure, left ventricular failure, stroke, cerebral vasospasm after subarachnoid injury, atherosclerotic heart disease, and retinal hemorrhage), renal disorders (e.g., renal vein thrombosis, kidney infarction, renal artery embolism, renal artery stenosis, and edema, hydronephritis), proliferative diseases or disorders (e.g., vascular stenosis, myocardial hypertrophy, hypertrophy and/or hyperplasia of conduit and/or resistance vessels, myocyte hypertrophy, and fibroblast proliferative diseases), inflammatory diseases (e.g., SIRS (systemic Inflammatory Response Syndromes), sepsis, polytrauma, inflammatory bowl disease, acute and chronic pain, rheumatoid arthritis, and osteo arthritis), allergic disorders (e.g., asthma, adult respiratory distress syndrome, wound healing, and scar formation), as well as several other disorders and/or diseases (e.g., periodontal disease, dysmenorrhea, premature labor, brain edema following focal injury, diffuse axonal injury, and reperfusion injury).

### FIGURES AND DRAWINGS

FIG. 1. SEQ ID NO:1. DNA sequence encoding scFv9B9.

FIG. 2. SEQ ID NO:2. Amino acid sequence of the scFv9B9 antibody.

FIG. 3. Phage ELISA on ACE-coated plates. Purified phages after 1^{st} round of selection were taken for analysis in ELISA. As a negative control to the scFv 9B9 we used scFv in which lambda light chain was substituted with non-specific aberrant kappa light chain. For that, 96 well plate was coated with human and rat ACEs as a positive controls, and bovine ACE and BSA (5 ug/ml) as a negative controls. Plates were blocked for 30 min with 2% non-fat dry milk, and phages diluted in the milk were applied to the plates. After 30 min incubation with shaking and another 1.5 hours without shaking, unbound phages were washed with PBS/0.05% Tween 20 and anti-M13 antibodies conjugated with peroxidase (Pharmacia Biotech) diluted 1/2000 dilution in the 2% non-fat dry milk were added. After intensive washing with PBS/0.05% Tween 20 plates were developed with 1-step 3,3′5,5′-Tetramethyl-benzidine (TBM) substrate for ELISA and read at OD620 or at OD450 after reaction was stopped with 3N HCl.

FIG. 4. Phage ELISA on ACE-expressing cells. (A) CHO cells line expressing human somatic ACE (clone 2C2) were grown in 96 well plate to confluency in HAM F12 medium supplemented with 10% FBS and 200 ug/ml geneticin. After washing with PBS cells were fixed with 4% paraformaldehyde (PFA) for 20 min at RT and stored at +4° C. until the use. ELISA with phages was performed as described for plate ELISA. (B) Rat lung microvascular endothelial cells (RLMVEC) were purchased from (VEC Technologies, Inc., Rensselaer, N.Y.). RLMVEC were grown to confluency in EBM-2 culture medium supplemented with growth factors on plates covered with 0.2% gelatin. Cells were processed for ELISA as described for CHO-ACE cells. (C) Rat lung microvascular endothelial cell line (RLMVEC) expressing human somatic ACE clone (1C10) were grown to confluency in DMEM culture medium supplemented with 10% FBS and 200 ug/ml geneticin. Cells were processed for ELISA as described for CHO-ACE cells.

FIG. 5. In vivo assay of specificity of scFv 9B9 phages to the lung vasculature. 9B9 scFv phages and their negative control scFv (where lambda was substituted with kappa light chain) were injected into rats for 30 minutes in titer ranging from 10⁹ to 10¹¹. After that rat's circulation was perfused through abdominal aorta with PBS until all blood was washed out. Organs were harvested, and homogenated in 5 ml PBS. Organs homogenates were used for titer determination of phages accumulated in organs. Ratio of lung to heart and lung to kidney was calculated as an index of specificity of lung accumulation. Lung to heart ratio for scFv 9B9 (lambda) exceeded that one for nonspecific scFv (kappa) more then 50 times. Lung to kidney ratio for scFv 9B9 exceeded that one for non-specific scFv (kappa) on average more then 6 times.

FIG. 6. ELISA on ACE-coated plates with scFv 9B9 as a soluble protein. Clone of XL1 blue E. coli transformed with pOPE101 expression vector carrying gene for scFv 9B9 was grown overnight in LB medium supplemented with 100 mM glucose and 100 ug/ml ampicilin (LBga) as previously described (Kipriyanov et al., 1996). Overnight cultures were diluted 1/100 and grown in 50 ml of LB_{GA} media until density ODeoo=0.8 with shaking 250 rpm at 37° C. After that bacterial cultures were centrifuged at 1500×g for 10 min, pellets were resuspended in 50 ml LB_{A} media containing 0.4 M sucrose and 0.1 mM IPTG and grown for 20 hours at 28° C. The culture supernatant and soluble periplasmic protein obtained according to protocol published by Kipriyanov et al., 1996 were directly used for analysis in Western blotting and for ELISA assay. 96 well plates coated with human, bovine ACE and BSA were blocked with 2% dry milk and supernatant and lysate were applied for 1 hour at RT. After washing, anti-c-myc monoclonal antibodies hybridoma supernatant (clone 9E10 from ATCC) diluted 1/30 was added with subsequent development of bound antibodies with anti-mouse antibodies conjugated with alkaline phosphatase and substrate. Reaction was read at OD₄₀₅.

FIG. 7. In vivo assay of specificity of scFv 9B9 (as a soluble protein) to the lung vasculature. Soluble scFv were purified from supernatant containing soluble scFv 9B9 using Ni-columns (Qiagen Inc, Valencia, Calif.). 100 ug of pure scFv 9B9 were labeled with 100 uCi of I¹²⁵ using Iodo-Gen tube (Pierce, Rockford, Ill.). Free iodine was removed using PG10 columns (GE Healthcare Bio-Sciences AB, Uppsala). I¹²⁵-labeled scFv 9B9 (1 mln cpm) was injected into rat's tail vein. In 1 hour animals were sacrificed and radioactivity of organs was counted in gamma counter. PHOG21 scFv was used as a negative control in the biodistribution study. Ratio of lung to heart and lung to blood was calculated as an index of specificity of lung accumulation I¹²⁵-labeled scFv 9B9. Lung to heart ratio for scFv 9B9 exceeded that one for non-specific scFv pHOG21 in 3.6 times. Lung to blood ratio for scFv 9B9 exceeded that one for non-specific scFv in 3.3 times.

FIG. 8. The Asn68Gln substitution in the heavy chain cDNA of scFv 9B9 improves binding ability of 9B9 scFv with human ACE. All antibodies contain carbohydrate at conserved positions in the constant regions of the heavy chains (Carayannopoulos L, and Capra J. D., 1993). Sequence analysis of cloned scFv 9B9 revealed the presence the site of N-glycosylation in its variable region: Asn68 together with Ile69 and Thr70 forms the site of N-glycosylation (NIT) in FR3 region of heavy chain in near proximity of CDR2. It is known that antibodies produced in prokaryotic cells are not glycosylated (Matsuda h et al., 1990). In order to investigate the effect of glycosylation scFv 9B9 on its binding activity with ACE, scFv 9B9 was re-cloned from prokaryotic expression vector pOPE101 into mammalian expression vector pSecTag2 (Invitrogen, Inc) and the site of glycosylation was removed by mutation of Asn68 to Gln68. Both constructs, 9B9scFv in pSecTag2 and 9B9scFv (N68Q) in pSecTag2, were transfected into CHO cells and supernatants containing 9B9scFv and 9B9scFv (N68Q) were analyzed for their binding with human ACE expressed on the surface of CHO cells (Balyasnikova et al., 1999). 9B9scFv (N68Q) showed on average 2.7 times (ranged between 1.7 and 3.4) higher binding with human ACE expressed on the surface of CHO cells then 9B9scFv. Thus, the mutation of Asn68 to Gin in 9B9scFv c DMA significantly improved the ability of 9B9 scFv to interact with human ACE.

FIG. 9. Cell surface expression of scFv 9B9. The expression of 9B9scFv on the surface of the cells could be necessary when the delivery of certain cell types to the pulmonary circulation is desirable. Examples might include but not limited to by delivery of dendritic or steam cells to the pulmonary vasculature. 9B9 scFv or its N68Q mutant variant were sub-cloned in pDisplay vector (Invitrogen Inc.) using SfiI and Accl restriction sites. For that, 9B9sc Fv was amplified with a pair of primer corresponding to N and C terminal sequence of 9B9sc Fv from pSex81 as template followed by re-amplification with a set of primers introducing SfiI restriction site to its N-terminai sequence and Accl restriction site to its C-terminal sequence. The obtained constructs were amplified, purified and transfected into HEK cells using Lipofectamin Plus reagent from Invitrogen Inc. In 48 hours cells were collected using cell dissociation solution (Gibco), centrifuged and 1.5×10⁶ cells were lysed in 200 ul of 8 mM CHAPS. 10 ul of lysate of HEK cells alone (1), HEK cells expressing 9B9scFv (2), HEK cells expressing 9B9scFv N68Q mutant variant (3) or soluble 9B9scFv (4) produced in E. coli were applied to 10% Bio-Red gel and run in reducing condition. After transfer to the membrane, the proteins were reveled by anti c-myc monoclonal antibodies (clone 9E10 from ATCC) followed by development with secondary anti-mouse antibody-biotin conjugate and streptavidin-peroxidase and West Pico (Pierce) development chemiluminescent reagent.

FIG. 10. Summary data for FIG. 4A-C. Binding of phages harboring the λ 9B9 short chain to CHO cell line expressing human somatic angiotensin converting enzyme as compared to hACE-expressing RLMVEC and ratACE-expressing RLMVEC-ratACE.

FIG. 11. Production of scFv 9B9 (A) and scFv9B9 N68Q (B) by CHO cells at 30° C. versus 37° C. CHO cells were transiently transfected with a plasmid pSecTaq 2 encoding scFv 9B9 or scFv 9B9 N68Q using standard transfection protocol and Lipofectamin Plus reagent from Invitrogen Inc. In 24 hours cells were cultivated either at 30° C. or 37° C. in atmosphere containing 5% CO₂. Culture medium was replaced with fresh culture medium every two days during two weeks. Collected samples were frozen in liquid nitrogen and kept at −80° C. until assayed. The binding of antibody fragments was estimated in ELISA using CHO-hACE (clone 2C2) cells. To confirm that the increased binding in ELISA is due to increased production of antibody fragments at 30° C., the samples of 9B9scFvN68Q collected at day 2 and day 5 from the cells incubated at 30° C. were assayed by Western Blotting (blot not shown).

FIG. 12. The binding 9B9scFvN68Q human Fc fusion to CHO-hACE (clone 2C2) cells. Human Fc fragment (hinge, CH2 and CH3 regions) was cloned using the set of gene specific primers from the cDNA obtained from mononuclear fraction of blood of healthy volunteer. The primers are identified herein as SEQ ID NO:21, SEQ ID NO:22, SEQ ID NO:23, and SEQ ID NO:24. Pair of restriction sites was introduced to the ends of cloned Fc fragment by PCR reaction for its subsequent re-cloning between 9B9scFv N68Q DNA and myc epitope in pSecTaq 2 plasmid. CHO cells were transiently transfected with generated fusion DNA. In 48 hours supernatant containing 9B9 scFvN68Q hFc secreted fusion was collected and its functionality was assayed in ELISA on CHO-hACE cells versus 9B9 scFvN68Q. The bound fragments were revealed by anti-myc antibody or by anti-human Fc fragment specific antibody (Sigma) conjugated with alkaline phosphatase.

FIG. 13. The specific transduction of RLMVEC-hACE by chimeric virus (9B9scFv). RLMVEC and RLMVEC-hACE were infected with control or chimeric virus encoding Lac z reporter gene packaged at 30° C. or 37° C. 48 hours post-infections cells were stained for X-gal activity and number X-gal positive cells (TU) was calculated for conrol and chimeric virus in each cell line. The specificity of transduction of RLMVEC-hACE by chimeric virus was estimated as ratio of its TE to TE of control virus.

FIG. 14. Sequences of the present invention.

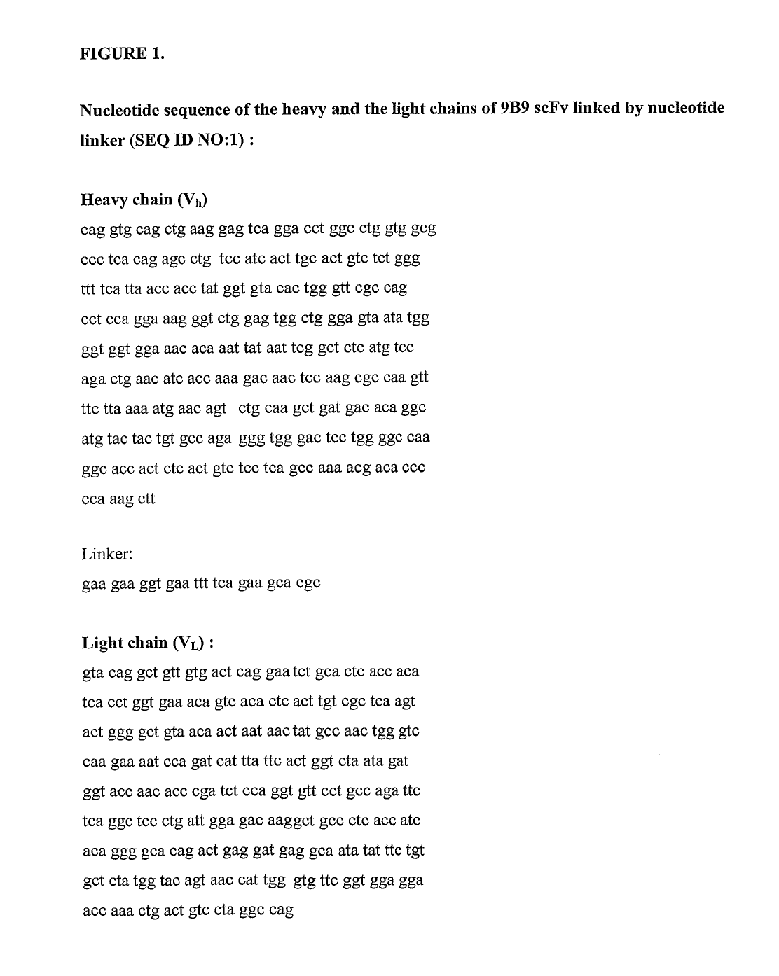

### DEFINITIONS

A single chain Fv fragment (scFv) is the smallest antibody fragment which retains antigen-binding capacity of the parental antibody. scFv consists of the variable regions of the heavy (V_{H}) and the light chains (V_{L}), which are linked through a flexible peptide linker. Variable regions comprise the amino-terminal domain of both heavy and light chains (Carayannapoulos and Capra, 1993). The affinity of single-chain fragments may be compromised due to its monovalent nature, however their avidity can be significantly increased by engineering scFv into multimers (from bi-to tetravalent molecules).

A polynucleotide can be delivered to a cell to express an exogenous nucleotide sequence, to inhibit, eliminate, augment, or alter expression of an endogenous nucleotide sequence, or to affect a specific physiological characteristic not naturally associated with the cell. The polynucleotide can be a sequence whose presence or expression in a cell alters the expression or function of cellular genes or RNA. A delivered polynucleotide can stay within the cytoplasm or nucleus apart from the endogenous genetic material. Alternatively, DNA can recombine with (become a part of) the endogenous genetic material. Recombination can cause DNA to be inserted into chromosomal DNA by either homologous or non-homologous recombination.

The term “active agent” is meant to refer to compounds that are therapeutic agents or imaging agents.

The term “therapeutic agent” and/or “selected drug” is meant to refer to any agent having a therapeutic effect, including but not limited to chemotherapeutics, toxins, radiotherapeutics, or radiosensitizing agents.

The term “chemotherapeutic” is meant to refer to compounds that, when contacted with and/or incorporated into a cell, produce an effect on the cell, including causing the death of the cell, inhibiting cell division or inducing differentiation.

The term “toxin” is meant to refer to compounds that, when contacted with and/or incorporated into a cell, produce the death of the cell.

The term “radiotherapeutic” is meant to refer to radionuclides which when contacted with and/or incorporated into a cell, produce the death of the cell.

The term “radiosensitizing agent” is meant to refer to agents which increase the susceptibility of cells to the damaging effects of ionizing radiation or which become more toxic to a cell after exposure of the cell to ionizing radiation. A radiosensitizing agent permits lower doses of radiation to be administered and still provide a therapeutically effective dose.

The term “imaging agent” is meant to refer to compounds which can be detected.

By “gene” it is meant a nucleic acid that encodes an RNA, for example, nucleic acid sequences including but not limited to structural genes encoding a polypeptide.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123 133; Frier et al., 1986, Proc. Nat. Acad. Sci. USA 83:9373 9377; Turner et al., 1987, J. Am. Chem. Soc. 109:3783 3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

The term “recombinant” is used to describe non-naturally altered or manipulated nucleic acids, host cells transfected with exogenous nucleic acids, or polypeptide molecules that are expressed non-naturally, through manipulation of isolated nucleic acid (typically, DNA) and transformation or transfection of host cells. “Recombinant” is a term that specifically encompasses nucleic acid molecules that have been constructed in vitro using genetic engineering techniques, and use of the term “recombinant” as an adjective to describe a molecule, construct, vector, cell, polypeptide or polynucleotide specifically excludes naturally occurring such molecules, constructs, vectors, cells, polypeptides or polynucleotides.

The term “bacteriophage” is defined as a bacterial virus containing a nucleic acid core and a protective shell built up by the aggregation of a number of different protein molecules. The terms “bacteriophage” and “phage” are synonymous and are used herein interchangeably.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g. in an in vitro transcription/translation system or in a host cell when the vector is introduced into the host cell). The term “regulatory sequence” is intended to included promoters, enhancers, and other expression control elements (e.g. polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g. tissue-specific regulatory sequences). It will be appreciated by those skilled in the art that the design of the expression vector can depend on such factors as the choice of the host cell to be transformed, the level of expression of protein or RNA desired, etc. The expression vectors of the invention can be introduced into host cells to thereby produce siNAs, RNAs, proteins or peptides, including fusion proteins or peptides.

As used and understood herein, percent homology or percent identity of two amino acid sequences or of two nucleic acid sequences is determined using the algorithm of Karlin and Atschul (Proc. Natl. Acad. Sci. USA, 87: 2264-2268 (1990)), modified as in Karlin and Altschul (Proc. Natl. Acad. Sci. USA, 90: 5873-5877 (1993)). Such an algorithm is incorporated into the NBLAST and XBLAST programs of Altschul et al. (J. Mol. Biol., 215: 403-410 (1990)). BLAST nucleotide searches are performed with the NBLAST program to obtain nucleotide sequences homologous to a nucleic acid molecule described herein. BLAST protein searches are performed with the XBLAST program to obtain amino acid sequences homologous to a reference polypeptide. To obtain gapped alignments for comparison purposes, Gapped BLAST is utilized as described in Altschul et al. (Nucleic Acids Res., 25: 3389-3402 (1997)). When utilizing BLAST and Gapped BLAST programs, the default parameters of the respective programs (e.g., XBLAST and NBLAST) are used. See, http://www.ncbi.nlm.nih.gov. Alternatively, the percent identity of two amino acid sequences or of two nucleic acid sequences can be determined once the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The term “binding” refers to the determination by standard techniques that a binding polypeptide recognizes and binds to a given target. Such standard techniques include, but are not limited to, affinity chromatography, equilibrium dialysis, gel filtration, enzyme linked immunosorbent assay (ELISA), FACS analysis, and the monitoring of spectroscopic changes that result from binding, e.g., using fluorescence anisotropy, either by direct binding measurements or competition assays with another binder.

The term “epitopes” as used herein refers to portions of ACE having antigenic or immunogenic activity in an animal, preferably a mammal. An epitope having immunogenic activity is a portion of ACE that elicits an antibody response in an animal. An eptiope having antigenic activity is a portion of ACE to which an antibody or ACE binding polypeptide specifically binds as determined by any method known in the art, for example, by the immunoassays described herein. Antigenic epitopes need not necessarily be immunogenic.

In another embodiment, a nucleic acid of the invention is expressed in mammalian cells using a mammalian expression vector. The recombinant mammalian expression vector may be capable of directing expression of the nucleic acid preferentially in a particular cell type (e.g. tissue-specific regulatory elements are used to express the nucleic acid). Tissue specific regulatory elements are known in the art. Non-limiting examples of suitable tissue-specific promoters include the albumin promoter, lymphoid-specific promoters, neuron specific promoters, pancreas specific promoters, and mammary gland specific promoters. Developmentally-regulated promoters are also encompassed, for example the murine hox promoters and the α-fetoprotein promoter.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18^{th} Ed. (1990). Pharmaceutical carriers may be selected in accordance with the intended route of administration and the standard pharmaceutical practice. For example, formulations for intravenous administration may include sterile aqueous solutions which may also contain buffers or other diluents. Appropriate pharmaceutical vehicles can be routinely determined by those of skill in the art. By “animal” it is meant to include, but is not limited to, mammals, fish, amphibians, reptiles, birds, marsupials, and most preferably, humans. The ability of mAb 9B9 to cross-react with ACE in a number of different animals including human, monkey, rat, cat, and hamster ACE was demonstrated by Danilov et al. in International Immunology (1994) 6(8):1153-1160.

### DETAILED DESCRIPTION OF THE INVENTION

The present invention relates to compounds and methods which are useful in the diagnosis, prevention, and therapy of diseases associated with lung tissue. These compounds are stable nucleic acid agents, or their expressed counterparts, which may be used to direct treatment to specific lung tissue. An example of one such nucleic acid agent is identified as SEQ ID NO:1.

An example of one such protein molecule is the herein described scFv9B9 antibody (SEQ ID NO:2).

In one embodiment, the present invention discloses an isolated DNA encoding a single chain fragment of the monoclonal antibody 9B9. Antibody fragments that recognize specific eptiopes may be generated by known techniques. Examples of techniques which can be used to produce single-chain Fvs and antibodies include those described in U.S. Pat. Nos. 4,946,778 and 5,458,498; Huston et al. methods in enzymology, 203:46-88 (1991); Shu et al., Proc. Natl. Acad. Sci. USA, 90:7995-7999 (1993); and Skerra et al., Science, 240:1038-1040 91988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science, 229:1202 (1985); Oi et al., BioTechniques, 4:214 (1986); Gillies et al., J. Immunol. Methods, 125:191-202 (1989); U.S. Pat. Nos. 5,807,715; 4,816,567; and 4,816,397, which are incorporated herein by reference in their entirety. A humanized antibody is an antibody molecule made using one or more complementarity determining regions (CDRs) from a non-human species antibody that binds the desired antigen and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature, 332:323 (1988), which are incorporated herein by reference in their entireties.) Antibodies can be humanized using a variety of techniques known in the art including, for example, CDR-grafting (EP 239 400; PCT publication WO 91/09967; U.S. Pat. Nos. 5,225,539; 5,530,101; and 5,585,089), veneering or resurfacing (EP 592 106; EP 519 596; Padlan, Molecular Immunology, 28(4/5):489-498 (1991); Studnicka et al., Protein Engineering, 7(6):805-814 (1994); Roguska. et al., Proc. Natl. Acad. Sci. USA, 91:969-973 (1994)), and chain shuffling (U.S. Pat. No. 5,565,332).

The sequences disclosed herein may be manipulated using methods well known in the art, e.g. recombinant DNA techniques, site directed mutagenesis, PCR, etc. (see, for example, the techniques described in Sambrook et al., Molecular Cloning: A Laboratory Manual, 2d Ed. (Cold Spring Harbor Laboratory, Cold Spring Harbor, N.Y. 1990) and Current Protocols in Molecular Biology, Ausubel et al., eds. (John Wiley & Sons, NY 1993), which are both incorporated by reference herein in their entireties), to generate antibodies having a different amino acid sequence, for example to create amino acid substitutions, deletions, and/or insertions.

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody or portion thereof (preferably containing the heavy or light chain variable domain) of the invention has been obtained, the vector for the production of the antibody molecule may be produced by recombinant DNA technology using techniques well known in the art. Thus, methods for preparing a protein by expressing a polynucleotide containing an antibody-encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT publication WO 86/05807; PCT publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In one embodiment, the scFv 9B9 antibodies of the present invention comprise a heavy chain variable region linked to a light chain variable region via a flexible linker. Examples of flexible linkers include, but are not limited to, (Gly₄Ser)₂ (SEQ ID NO:16); (Gly₄Ser)₃ (SEQ ID NO:15); and (Gly₄Ser) (SEQ ID NO:17). Nucleotide sequence SEQ ID NO:13 encodes amino acid sequence SEQ ID NO:16. Nucleotide sequence SEQ ID NO:12 encodes amino acid sequence SEQ ID NO:15. Nucleotide sequence SEQ ID NO:14 encodes amino acid sequence SEQ ID NO:17. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene, 45:101 (1986); Cockett et al., Bio/Technology, 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J., 2:1791 (1983)), in which the antibody coding sequence may be ligated individually into the vector in frame with the lacZ coding region so that a fusion protein is produced; pIN vectors (Inouye & Inouye, Nucleic Acids Res., 13:3101-3109 (1985); Van Heeke & Schuster, J. Biol. Chem., 24:5503-5509 (1989)); and the like pGBX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. See, e.g., Logan & Shenk, Proc. Natl. Acad. Sci. USA, 81:355-359 (1984). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, Bittner et al., Methods in Enzymol., 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, NSO, 293, 3T3, W138, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell, 11:223 (1977)), hypoxanthine-guanine phosphoribosyltransferase (Szybalska & Szybalski, Proc. Natl. Acad. Sci. USA, 48:202 (1992)), and adenine phosphoribosyltransferase (Lowy et al., Cell, 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Proc. Natl. Acad. Sci. USA, 77:357 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418; Wu and Wu, Biotherapy, 3:87-95 (1991); Tolstoshev, Ann. Rev. Pharmacol. Toxicol., 32:573-596 (1993); Mulligan, Science, 260:926-932 (1993); and Morgan and Anderson, Ann. Rev. Biochem., (1993); May, 1993, TIB TECH 11(5); 155-215); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Current Protocols in Molecular Biology, Ausubel et al., eds. (John Wiley & Sons, NY 1993); Kriegler, Gene Transfer and Expression, A Laboratory Manual (Stockton Press, NY 1990); and Current Protocols in Human Genetics, Dracopoli et al., eds. (John Wiley & Sons, NY 1994), Chapters 12 and 13; Colberre-Garapin et al., J. Mol. Biol., 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol., 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 322:52 (1986); Kohler, Proc. Natl. Acad. Sci. USA, 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalent and non-covalent conjugations) to a polypeptide (or, portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than ACE binding polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett., 39:91-99 (1994); U.S. Pat. No. 5,474,981; Gillies et al., Proc. Natl. Acad. Sci. USA, 89:1428-1432 (1992); Fell et al., J. Immunol., 146:2446-2452 (1991), which are incorporated by reference in their entireties.

The present invention further includes compositions comprising therapeutic or diagnostic polypeptides fused or conjugated to the scFv antibody domains. Methods for fusing or conjugating the polypeptides to antibody portions are known in the art. See, e.g., U.S. Pat. Nos. 5,336,603; 5,622,929; 5,359,046; 5,349,053; 5,447,851; 5,112,946; EP 307 434; EP 367 166; PCT publications WO 96/04388; WO 91/06570; Ashkenazi et al., Proc. Natl. Acad. Sci. USA, 88:10535-10539 (1991); Zheng et al., J. Immunol. 154:5590-5600 (1995); and Vil et al., Proc. Natl. Acad. Sci. USA, 89:11337-11341 (1992) (said references incorporated by reference in their entireties).

As discussed, supra, the polypeptides corresponding to an ACE binding polypeptide of the invention may be fused or conjugated to the above antibody portions to increase the in vivo half life of the polypeptides or for use in immunoassays using methods known in the art. Further, the ACE binding polypeptides of the invention may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394 827; Traunecker et al., Nature, 331:84-86 (1988). Moreover, the antibodies or fragments thereof of the present invention can be fused to marker sequences, such as a peptide to facilitate purification. In preferred embodiments, the marker amino acid sequencd is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad., Sci. USA, 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell, 37:767 (1984)) and the “flag” tag.

The present invention further encompasses a scFv 9B9 antibody conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In, or ⁹⁹Tc.

Further, the antibody scFv 9B9 may be conjugated to a therapeutic moiety such as a cytotoxin, e.g., a cytostatic or cytocidal agent, a therapeutic agent or a radioactive metal ion, e.g., alpha-emitters such as, for example, ²¹³Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (See, PCT publication WO 97/33899), AIM II (See, PCT publication WO 97/34911), Fas Ligand (Takahashi et al., Int. Immunol., 6:1567-1574 (1994)), VEGI (See, PCT publication WO 99/23105), CD40 Ligand, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), a plasminogen activator, a catalase, a superoxide dismutase, granulocyte macrophage colony stimulating factor (“GM-CSF”), granulocyte colony stimulating factor (“G-CSF”), or other growth factors. Examples of plasminogen activators include, but are not limited to, tissue-type PA (t-PA), urokinase PA (u-PA), and streptokinase.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al., eds. (Alan R. Liss, Inc. 1985), pp. 243-56; Hellstrom et al., “Antibodies For Drug Delivery”, in Controlled Drug Delivery (2nd Ed.), Robinson et al., eds. (Marcel Dekker, Inc. 1987), pp. 623-53; Thorpe, “Antibody Carriers Of Cytotoxic Agents In Cancer Therapy: A Review”, in Monoclonal Antibodies '84: Biological And Clinical Applications, Pinchera et al., eds., pp. 475-506 (1985); “Analysis, Results, And Future Prospective Of The Therapeutic Use Of Radiolabeled Antibody in Cancer Therapy”, in Monoclonal Antibodies For Cancer Detection And Therapy, Baldwin et al., eds. (Academic Press 1985), pp. 303-16; and Thorpe et al., “The Preparation And Cytotoxic Properties Of Antibody-Toxin Conjugates”, Immunol. Rev., 62:119-58 (1982).

Antibodies may also be attached to solid supports, which are particularly useful for immunoassays or purification of the ACE binding polypeptide. Such solid supports include, but are not limited to, glass, cellulose, polyacrylamide, nylon, polystyrene, polyvinyl chloride or polypropylene.

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

The present invention is further directed to antibody-based therapies which involve administering antibodies of the invention to an animal, preferably a mammal, and most preferably a human, patient for treating one or more of the diseases, disorders, or conditions disclosed herein. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant ACE expression and/or activity, including, but not limited to, any one or more of the diseases, disorders, or conditions described herein.

A composition is said to be a “pharmaceutically acceptable carrier” if its administration can be tolerated by a recipient patient. Sterile phosphate-buffered saline is one example of a pharmaceutically acceptable carrier. Other suitable carriers are well-known in the art. See, for example, REMINGTON'S PHARMACEUTICAL SCIENCES, 18^{th} Ed. (1990). Pharmaceutical carriers may be selected in accordance with the intended route of administration and the standard pharmaceutical practice. For example, formulations for intravenous administration may include sterile aqueous solutions which may also contain buffers or other diluents. Appropriate pharmaceutical vehicles can be routinely determined by those of skill in the art. By “animal” it is meant to include, but is not limited to, mammals, fish, amphibians, reptiles, birds, marsupials, and most preferably, humans. The ability of mAb 9B9 to cross-react with ACE in a number of different animals including human, monkey, rat, cat, and hamster ACE was demonstrated by Danilov et al. in International Immunology (1994) 6(8):1153-1160.

The treatment and/or prevention of diseases, disorders, or conditions associated with aberrant expression and/or activity of ACE or an ACE substrate includes, but is not limited to, alleviating symptoms associated with those diseases, disorders or conditions. The antibodies of the invention may also be used to target and kill cells expressing ACE on their surface and/or cells having ACE bound to their surface. This targeting may be the result of binding of the antibody to ACE binding polypeptides of the invention that have been coadministered, or alternatively, the result of direct binding of the antibody to ACE. Antibodies of the invention may be provided in pharmaceutically acceptable compositions as known in the art or as described herein.

Non-limiting examples of the ways in which the antibodies of the present invention may be used therapeutically includes binding ACE binding polypeptides that have been coadministered in order to bind or neutralize ACE, or by direct cytotoxicity of the antibody, e.g., as mediated by complement (CDC) or by effector cells (ADCC). ACE binding polypeptides and anti-ACE binding polypeptide antibodies may be administered either locally or systemically. Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy, anti-tumor agents, antibiotics, and immunoglobulin). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against ACE polypeptides of the present invention, fragments or regions thereof, for both immunoassays directed to and therapy of disorders related to ACE polypeptides, including fragments thereof, of the present invention. Such antibodies, fragments, or regions, will preferably have an affinity for polypeptides of the invention, including fragments thereof. Preferred binding affinities include those with a dissociation constant or K_{D} less than 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴M, 10⁻¹⁴ M, 5×10⁻¹⁵ M, and 10⁻¹⁵ M.

Labeled antibodies, and derivatives and analogs thereof, which specifically bind to an ACE binding polypeptide of interest can be used for diagnostic purposes to detect, diagnose, or monitor diseases and/or disorders associated with the aberrant expression and/or activity of ACE. The invention provides for the detection of aberrant expression of ACE, comprising (a) contacting cells or body fluid with an ACE binding polypeptide; (b) assaying the expression of ACE in cells or body fluid of an individual using one or more antibodies specific to the ACE binding polypeptide and (c) comparing the level of ACE expression with a standard ACE expression level, whereby an increase or decrease in the assayed ACE expression level compared to the standard expression level is indicative of aberrant expression.

The invention provides a diagnostic assay for diagnosing a disorder, comprising (a) contacting cells or body fluid with an ACE binding polypeptide; (b) assaying the expression of ACE in cells or body fluid of an individual using one or more antibodies specific to the ACE binding polypeptide of interest and (c) comparing the level of ACE expression with a standard ACE expression level, whereby an increase or decrease in the assayed ACE expression level compared to the standard expression level is indicative of a particular disorder. With respect to cancer, the presence of a relatively high amount of ACE in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay ACE protein levels in a biological sample using or routinely modifying classical immunohistological methods known to those of skill in the art (e.g., see Jalkanen et al., J. Cell. Biol., 101:976-985 (1985); Jalkanen et al., J. Cell. Biol., 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, 149 Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru; luminescent labels, such as luminol; and fluorescent labels, such as fluorescein and rhodamine, and biotin.

Techniques known in the art may be applied to label antibodies of the invention. Such techniques include, but are not limited to, the use of bifunctional conjugating agents (see, e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the contents of each of which are hereby incorporated by reference in its entirety).

One embodiment of the invention is the detection and diagnosis of a disease or disorder associated with aberrant expression of ACE in an animal, preferably a mammal and most preferably a human. In one embodiment, diagnosis comprises: (a) administering (for example, parenterally, subcutaneously, or intraperitoneally) to a subject an effective amount of a labeled molecule which specifically binds to ACE or which specifically binds to a molecule that specifically binds to ACE (e.g., an anti-ACE binding scFv antibody of the invention); (b) waiting for a time interval following the administering for permitting the labeled molecule to preferentially concentrate at sites in the subject where the ACE is expressed (and for unbound labeled molecule to be cleared, to background level); (c) determining background level; and (d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood by those skilled in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain the specific polypeptide. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds. (Masson Publishing Inc. 1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In a further embodiment, monitoring of the disease or disorder is carried out by repeating the method for diagnosing the disease or disorder, for example, one month after initial diagnosis, six months after initial diagnosis, one year after initial diagnosis, etc. and comparing the results.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include but are not limited to computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

In another specific embodiment, the present invention provides methods for amplifying the light chain fragment of the 9B9 antibody. The present invention further discloses a method of preparing single fragment 9B9 and cloning the same into a phagemid vector. As illustrated below, the phage expressing the scFv selectively bind endothelial cells expressing angiotensin converting enzyme (ACE).

In one embodiment the present invention provides methods for isolating and sequencing the light chain fragment of the 9B9 antibody, which is required to design efficient primers for amplification of the fragment. Analysis of the light chain fragment revealed that it is a λ type light chain. In one embodiment the present invention provides a method for phage ELISA on angiotensin converting enzyme coated plates. Purified phages expressing the single chain fragment of the antibody 9B9 were found to bind to human angiotensin converting enzyme and to some extent to rat angiotensin converting enzyme. No phage bound to bovine angiotensin converting enzyme or to BSA.

As described below, phages expressing the single chain fragment of the antibody 9B9 bind CHO cell lines expressing human somatic angiotensin converting enzyme (clone 2C2). The binding of the phages to rat lung microvascular endothelial cells is also demonstrated. Furthermore these phages were also shown to bind rat lung microvascular endothelial cells expressing human somatic angiotensin converting enzyme clone (1C10). Highest binding of phages was observed for the CHO cell lines expressing human somatic angiotensin converting enzyme (clone 2C2).

The present invention also discloses an in vivo assay in rats that illustrates the specificity of the single chain 9B9 antibody expressing phages for lung vasculature.

In another embodiment, the present invention uses various isolated DNAs encoding a single chain fragment of the monoclonal antibody 9B9 including, but not limited to, (a) isolated DNA which encodes a single chain fragment of the monoclonal antibody 9B9; (b) isolated recombinant DNA which hybridizes under high stringency conditions to isolated DNA of (a) above and which encodes a single chain fragment of the monoclonal antibody 9B9; (c) isolated DNA differing from the isolated DNAs of (a) and (b) above in codon sequence due to the degeneracy of the genetic code, and which encodes a single chain fragment of the monoclonal antibody 9B9. The isolated DNA preferably comprises a polynucleotide sequence of SEQ ID NO:1. In another embodiment, the isolated DNA preferably comprises a polynucleotide sequence of SEQ ID NO:19.

In another embodiment the present invention provides for expression of scFv 9B9 by preparing a vector construct with the isolated DNA encoding scFV 9B9 antibody. This vector may further comprise regulatory elements required for expression of the scFv 9B9 antibody. This vector may be, for example, a plasmid, a cosmid, a phagemid, a BAC or a YAC. A host cell such as a mammalian, plant or insect cell may be transfected with the vector of the present invention to express the scFv 9B9 antibody. Thus in a further embodiment, the present invention provides methods for directing cells bearing such vector constructs to the lung vasculature. Progenitor and stem cells required to replace damaged lung tissue can be directed to the lungs using such methods.

In another embodiment of the present invention the vector designed to express the scFv 9B9 antibody can further comprise other segments of DNA and regulatory elements necessary to express a therapeutic protein. This therapeutic protein is preferably a protein required by the lung of a mammal. As the lung vasculature is rich in angiotensin converting enzyme, this vector can be used to direct the therapeutic protein to the lung vasculature.

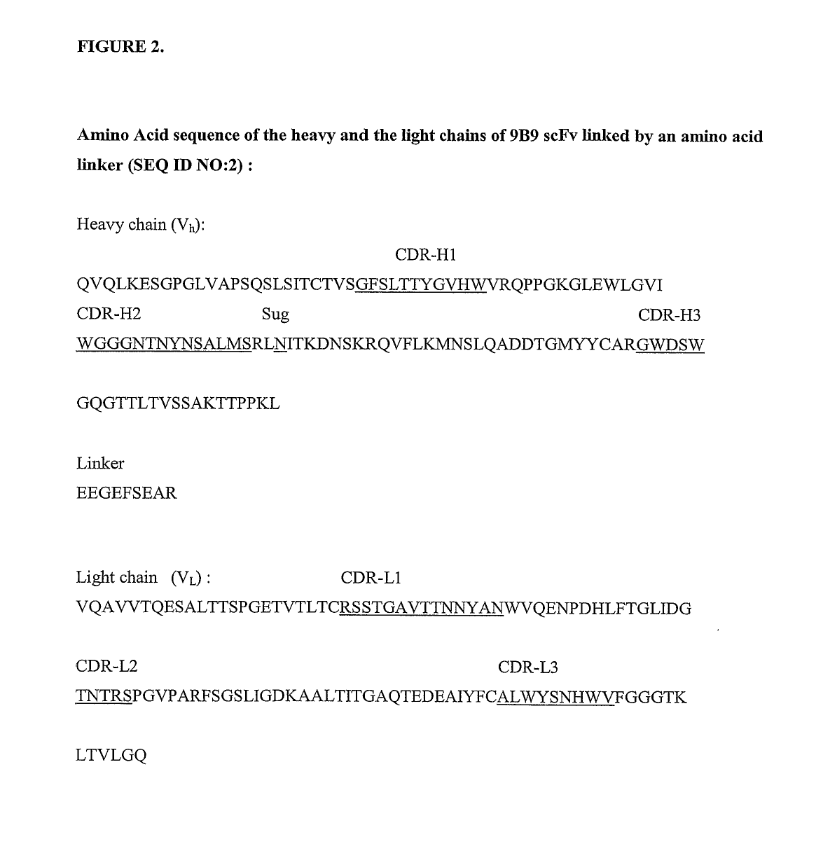

The instant invention further discloses the amino acid sequence of the scFv 9B9 antibody in FIG. 2 (SEQ ID NO:2). The instant invention also further discloses the amino acid sequence of a scFv 9B9 (N68Q) antibody (SEQ ID NO:18). These proteins may be conjugated with a therapeutic agent or diagnostic agent, which is to be delivered to the lung of a mammal. Thus in one embodiment the instant invention provides a method for treating a lung malady by administering a conjugate of the scFv protein and a therapeutic agent to a mammal in need of the therapeutic agent. In a further embodiment, the invention provides for diagnosis of a lung malady by administering a conjugate of the scFv protein and a diagnostic agent.

The instant invention further discloses a set of isolated DNA fragments that encode different polymers of the scFv protein. It is contemplated that vectors comprising these DNA fragments can be prepared such that by transfecting host cells with such vectors different polymers of the scFv protein can be expressed in the host cell. This vector may be a plasmid, a cosmid, a phagemid, a BAC or a YAC. A host cell such as a mammalian, plant or insect cell may be transfected with the vector of the present invention to express different polymers of the scFv 9B9 antibody. Thus in one embodiment, the present invention provides a method for directing cells bearing such vector constructs to the lung vasculature. Progenitor and stem cells required to replace damaged lung tissue can be directed to the lungs using this method.

In one embodiment of the present invention vectors designed to express polymers of the herein disclosed scFv 9B9 antibodies can further comprise the DNA and regulatory elements required to express a therapeutic protein. This therapeutic protein is preferably a protein required by the lung of a mammal. As the lung vasculature is rich in angiotensin converting enzyme, this vector can be used to direct the therapeutic protein to the lung vasculature.

The instant invention further discloses a polymer of one or more of the herein disclosed scFv 9B9 antibodies. Preferably the polymer comprises about 2 to about four monomers of the scFv 9B9 antibody. These polymers known in the art as diabodies, tribodies etc, depending on the number of monomers present, may be conjugated with a therapeutic agent or diagnostic agent, which is to be delivered to the lung of a mammal. Thus in one embodiment the instant invention provides a method for treating a lung malady by administering a conjugate of a polymer of scFv 9B9 antibody, and a therapeutic agent to a mammal in need of the therapeutic agent. In a further embodiment, the invention provides for diagnosis of a lung malady by administering a conjugate of a polymer of the scFv 9B9 antibody and a diagnostic agent.

In one embodiment the invention teaches the expression of the single chain fragment of 9B9 in E. coli using a POPE vector carrying the gene for the single chain fragment of 9B9. The protein was secreted out of the cell as was seen from its presence mainly in the cell supernatant as compared to the cell lysate. Furthermore the invention discloses an in vivo assay that t demonstrates the accumulation of the protein in the lungs of rats.

For purposes of immunotherapy, an immunoconjugate and a pharmaceutically acceptable carrier are administered to a patient in a therapeutically effective amount. A combination of an immunoconjugate and a pharmaceutically acceptable carrier is said to be administered in a “therapeutically effective amount” if the amount administered is physiologically significant. An agent is physiologically significant if its presence results in a detectable change in the physiology of a recipient patient.

Additional pharmaceutical methods may be employed to control the duration of action of an immunoconjugate in a therapeutic application. Control release preparations can be prepared through the use of polymers to complex or adsorb an immunoconjugate. For example, biocompatible polymers include matrices of poly(ethylene-co-vinyl acetate) and matrices of a polyanhydride copolymer of a stearic acid dimer and sebacic acid. Sherwood et al., Bio/Technology 10:1446-1449 (1992). The rate of release of nucleic acid molecule from such a matrix depends upon the molecular weight of the molecule, the amount of molecule within the matrix, and the size of dispersed particles. Saltzman et al., Biophysical. J. 55:163-171 (1989); and Sherwood et al., Bio/Technology 10:1446-1449 (1992). Other solid dosage forms are described in REMINGTON'S PHARMACEUTICAL SCIENCES, 18th Ed. (1990).

Having now generally described the invention, the same will be more readily understood through reference to the following Examples which are provided by way of illustration, and are not intended to be limiting of the present invention. Isolation of scFv9B9 antibodies and their use in accordance with this invention are illustrated below and in the figures.

### Example 1

### Cloning and Isolation of Single Chain Fragment of 9B9 Antibody

The heavy chain of the monoclonal antibody 9B9 was obtained using a set of gene specific primers for heavy chain of immunoglobulin. To obtain the light chain fragment purified monoclonal antibody 9B9 was subjected to 2D electrophoresis. Several spots corresponding to the light chain were observed on the 2D gel. The major spot was chosen for extraction of protein and N-terminal sequence (Edman degradation). Analysis revealed that the light chain is a λ type light chain. Based on the amino acid sequence specific primers for the amplification of the light chain immunoglobulins from cDNA of the monoclonal antibody 9B9 was used.

Heavy and light chain sequences were subcloned into phagemid vector pSEX 81 and several rounds of selection were performed using human angiotensin-converting enzyme (ACE) absorbed on the plate for selection of immunoreactive phages. Bacterial clones infected with positively selected phages were screened by PCR reaction for presence of the right size PCR products (around 1000 base pairs) which consisted of scFv 9B9 and the vector's sequences upstream and downstream of the scFv 9B9 insert. Three PCR products in the range of 1000 base pairs were purified. The single chain fragment thus obtained was further analyzed by expression on phage surface and also by expressing it as a soluble protein using E. coli.

### Example 2

### Phage ELISA on ACE-Coated Plates

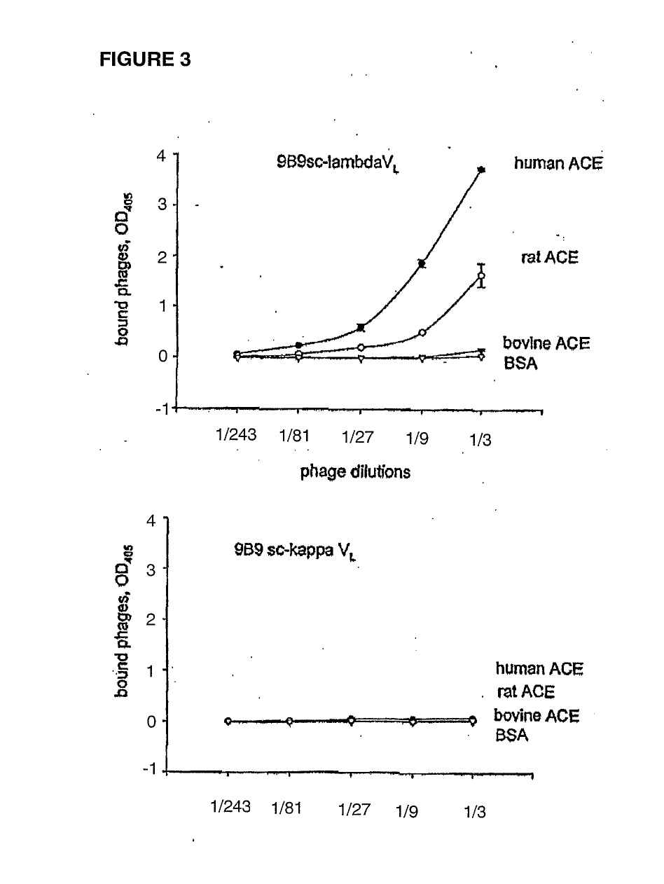

After the first round of selection, purified phages were analyzed for expression of scFv 9B9 using ELISA. As a negative control to the scFv 9B9, scFv in which the λ light chain was substituted with non-specific k light chain was used. For the assay 96 well plates were coated with human and rat ACEs as positive controls and with bovine ACE and BSA (5 μg/ml) as negative controls. Plates were blocked for 30 min with 2% non-fat dry milk and the phages diluted in milk were applied to the plates. After a two hour incubation period during which the plates were on a shaker for 30 minutes and kept stationary for the rest of the time, unbound phages were washed with PBS/0.05% Tween 20. Then anti-M13 antibodies conjugated with peroxidase (Amersham) diluted 1/2000 in milk was added. After Intensive washing with PBS/Tween 20 the plates were developed with 1-step TBM substrate for ELISA and read at 620 nm or 450 nm after the reaction was stopped with 3N HCl. There was substantial binding of phages with the λ fragment to plates coated with human angiotensin converting enzyme as compared to rat angiotensin converting enzyme (FIG. 3). These phages did not bind to bovine angiotensin converting enzyme or BSA. The phages with κ fragment did not bind to any of the angiotensin converting enzymes (FIG. 3). These results demonstrate the specificity of scFv 9B9 for human angiotensin converting enzyme and also show that the λ light chain is required for antigen binding.

### Example 3

### Phage ELISA on ACE-Expressing CHO Cell Line

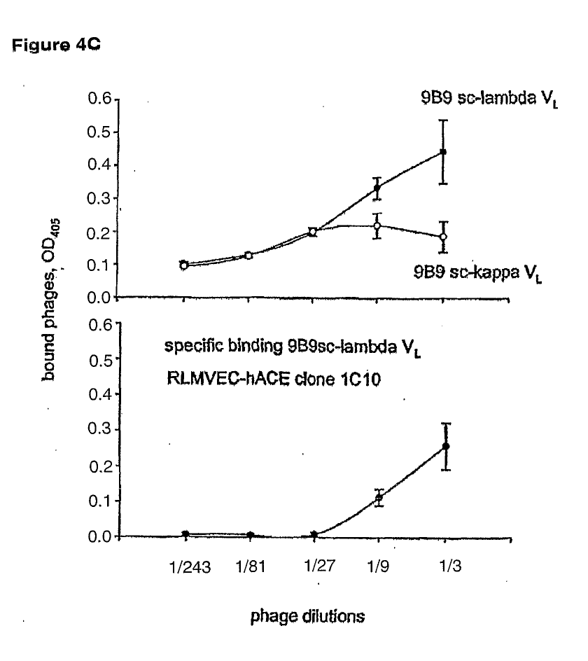

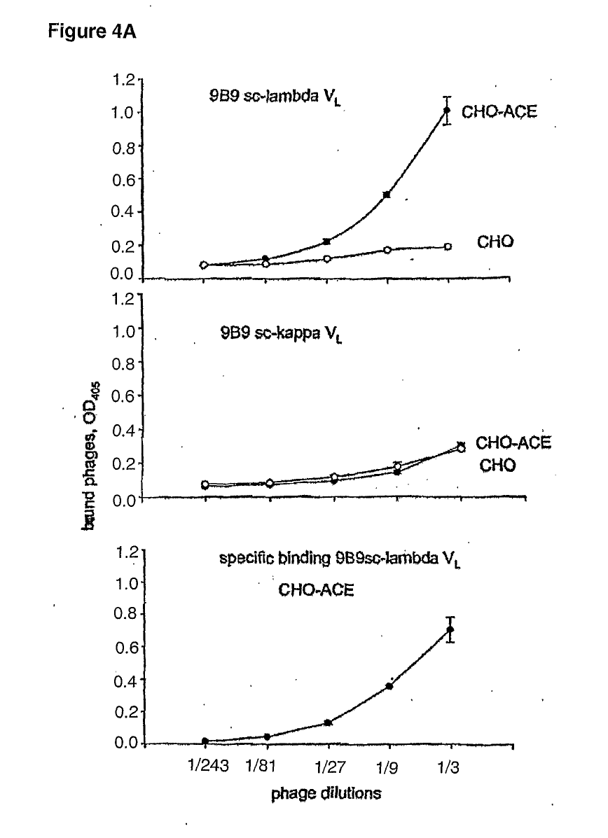

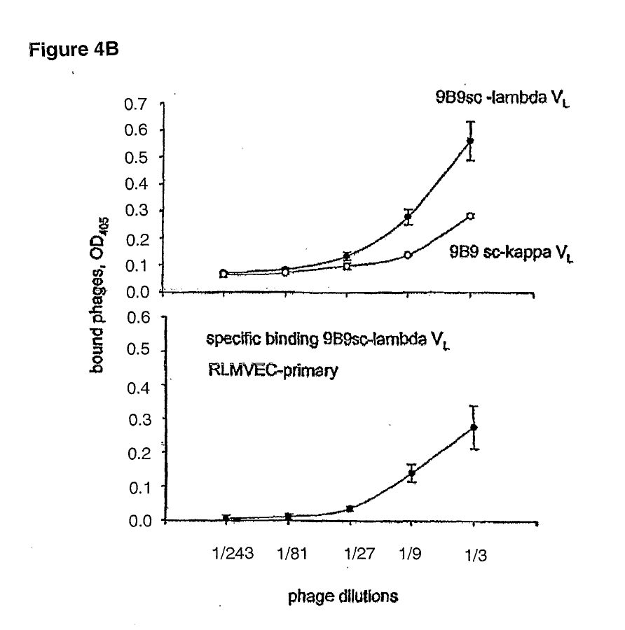

CHO cells line expressing human somatic angiotensin converting enzyme (clone 2C2) were grown in 96 well plate to confluence in HAM F12 medium supplemented with 10% FBS and 200 ng/ml genetkm After washing with PBS cells were fixed with 4% paraformaldehyde (PFA) for 20 minutes at room temperature and stored at 4° C. until further use. ELISA with phages was performed as described in example 2. FIG. 4A shows the excellent binding of phages with the λ fragment to the angiotensin converting enzyme expressing cells as compared to phages with the κ fragment.

### Example 4

### Phage ELISA on ACE-Expressing Rat Lung Microvascular Endothelial Cells (RLMVEC)

Rat lung microvascular endothelial cells (RLMVEC) was purchased from (VEC Technologies, Inc., Rensselaer, N.Y.). Rat lung microvascular endothelial cells were grown to confluency in EBM-2 culture medium supplemented with growth factors on plates covered with 0.2% gelatin. Cells were processed for ELISA as described in examples 2 and 3. The phages with λ fragment bound to the rat lung microvascular endothelial cells albeit to a lesser extent as compared to CHO cell line expressing human somatic angiotensin converting enzyme (FIGS. 4A and 4B).

### Example 5

### Phage ELISA on RMLVEC Cell Lines Expressing Human Somatic ACE

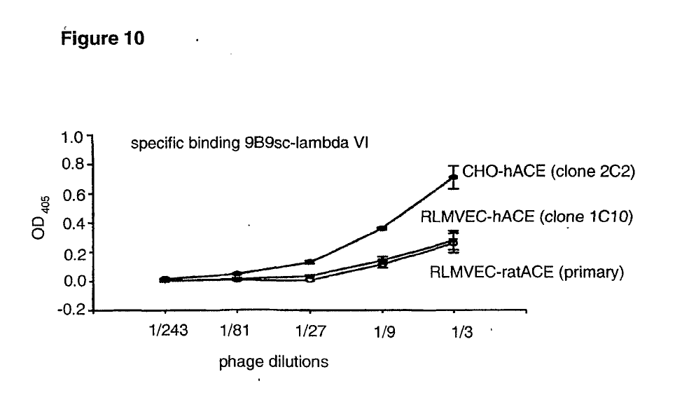

Rat lung microvascular endothelial cell line expressing human somatic angiotensin converting enzyme (clone 1C10) were grown to confluency in DMEM culture medium supplemented with 10% FBS and 200 ng/ml geneticin. Cells were processed for ELISA as described for CHO-ACE cells (FIG. 4C). The phages with λ fragment bound to the rat lung microvascular endothelial cells. FIG. 10 (summary data for FIG. 4A-C) illustrates the binding of phages with the λ fragment that was observed for CHO cell line expressing human somatic angiotensin converting enzyme as compared to hACE-expressing RLMVEC and ratACE-expressing RLMVEC-ratACE.

### Example 6

### In Vivo Assay of Specificity of scFv 9B9 Phages to the Lung Vasculature

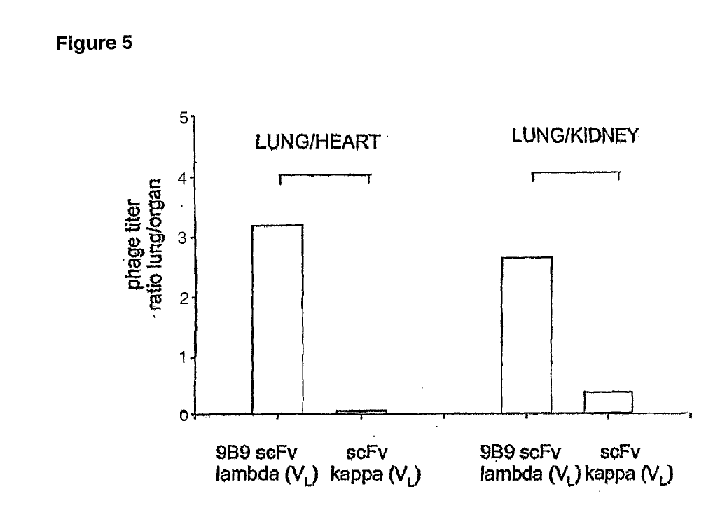

9B9 scFv phages and their negative control scFv (where λ was substituted with κ light chain) were injected into rats for 30 minutes (titer from 10⁹ to 10¹¹). Then the rat's circulation was perfused through abdominal aorta with PBS until all blood was washed out. Organs were harvested and homogenated in 5 ml PBS. Organ homogenates were used for titer determination of phages accumulated in different organs. Ratio of lung to heart and lung to kidney was calculated as an index of specificity of lung accumulation (FIG. 5). The lung to heart ratio for scFv 9B9 was over 50 times more than that for non-specific scFv (κ) and the lung to kidney ratio for scFv 9B9 was over 6 times more than that for non-specific scFv (κ).

### Example 7

### ELISA on ACE-Coated Plates with scFv as Soluble Protein

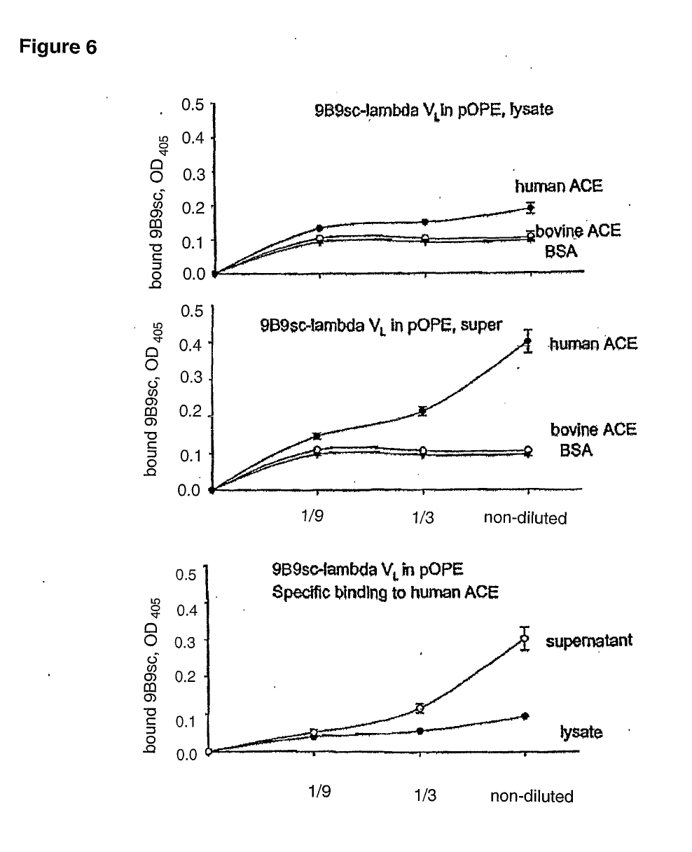

Clone of XL1 blue E. coli transformed with pOPE expression vector carrying the gene for scFv 9B9 was grown overnight in LB medium supplemented with 100 mM glucose and 100 ug/ml ampicilin, Overnight culture was diluted 1/100 and was grown to a density of 0.6 OD₆₀₀. After which the bacterial culture was centrifuged and cell pellet was resuspended in the same volume of LB media but with 0.4 M sucrose, 100 mM IPTG and 100 ug/ml ampicillin and grown overnight at 30° C. The supernatant from this culture was collected by centrifugation and bacterial pellet was lysed in lysis buffer. Both, supernatant and lysate containing soluble scFv were used for ELISA assay. 96 well plates coated with human or bovine ACE and BSA were blocked with 2% dry milk and the supernatant and lysate were applied to the plates for 1 hour at room temperature. After washing, anti-myc monoclonal antibodies hybridoma diluted 1/30 was added with subsequent development of bound antibodies with anti-mouse Ab-conjugated with alkaline phosphatase and the reaction was read at 405 nm (FIG. 6). The scFv 9B9 is mainly secreted out from the cells as is demonstrated by its higher concentration in the supernatant as compared to the lysate.

### Example 8

In Vivo Assay of Specificity of scFv 9B9 as a Soluble Protein to the Lung Vasculature

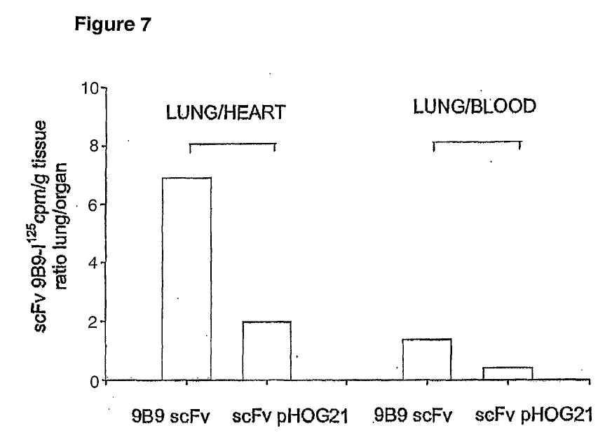

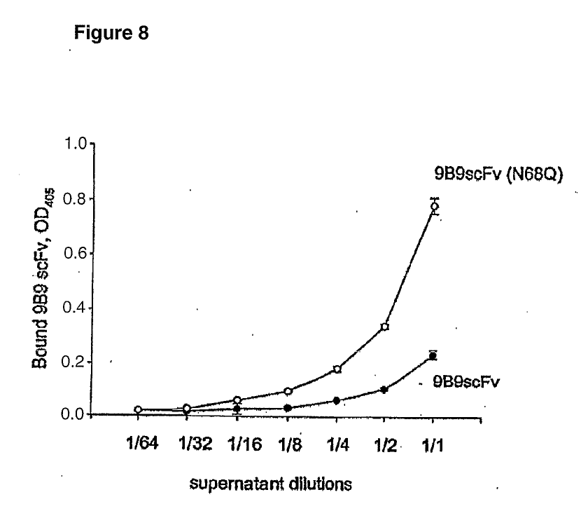

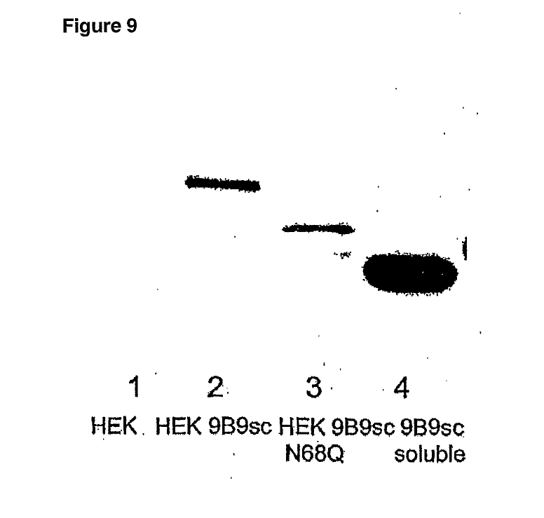

Soluble scFV protein was purified from the supernatant of XL1 blue E. coli transformed with pOPE expression vector carrying the gene for scFv 9B9 using Ni-columns (Qiagen). 100 μg of pure scFv 9B9 was labeled with 100 μCi of I¹²⁵ using Iodogen tube (Amersham). Free iodine was removed using PG10 columns. I¹²⁵ labeled scFv 9B9 (1 mln cpm) was injected into the rat's tail vein. An hour after the injection animals were sacrificed and radioactivity of organs was counted in gamma counter. PHOG21 scFv was used as a negative control in biodistribution study. Ratio of the amount of the soluble protein present in the lung to that present in the heart and the same ratio for the lung and blood were calculated as an index of specificity of lung accumulation (FIG. 7). The lung to heart ratio for scFv 9B9 was 3.6 times higher than the non specific PHOG21 scFv and the lung to blood ratio was 3.3 times higher than the non-specific PHOG21 scFv.

### Example 9

### Delivery of Therapeutics to the Lung Endothelial Cells

Viruses genetically modified by insertion of scFv 9B9 cDNA or cDNA encoding polymers of scFv 9B9 into the viral genome may be used to direct therapeutic genes to lung endothelial cells. This viral vector accumulates in the lung because the lung endothelium is rich in ACE.

### Example 10

### Delivery of Mammalian Cells to the Lung Endothelium

ScFv 9B9 cDNA or cDNA encoding polymers of scFv 9B9 may be used for transfection of mammalian cells. In this case the surface of such mammalian cells will express either scFv 9B9 antibody or a polymer thereof and will be directed to the lung endothelium. This provides a route for repairing damaged lung cells. These mammalian cells may also be transfected with therapeutic genes for treating a lung disease or disorder.

### Example 11

### Delivery of Therapeutic Proteins to the Lung Endothelium

Fusion constructs of the gene encoding a therapeutic protein and the scFv fragment (or a polymer of scFv 9B9) can be directed by a vector to the lung endothelium. The therapeutic protein will be expressed in the lung cells and mediate repair of lung cells. For example by directing catalase producing gene to the lung one can protect the lung endothelial cells from oxidative injury.

### Example 12

Nucleotide and Amino Acid Sequences of the Heavy and Light Chains of scFv9B9 Linked by Polypeptide Linker

The heavy chain of mAb 9B9 was obtained hybridoma cell line using the set of gene specific primers for heavy chain of immunoglobulin (Toleikis et al., 2004). The light chain was obtained in following manner. The set of lambda chain primers (MulgλV_{L}5′-A and MulgλV_{L}3′-1) from Novagen was used to amplify light chain fragment from cDNA 9B9. The obtained PCR product was sequenced using automatic sequencing technology. The nucleotide sequence was converted to amino acid format and was found to be matched to the amino-acid sequence obtained by Edman degradation of N-terminal part of light chain major spot extracted from 2D electrophoresis. (Briefly: purified mAb 9B9 was applied for 2D electrophoresis. Analysis revealed several spots corresponding to light chain. Major spot was chosen for extraction of protein and N-terminal sequence (Edman degradation). Analysis reveled that light chain belongs to the lambda type).

Based on the nucleotide sequence of PCR product obtained using pair of primers for lambda light chain from Novagen, primers suitable for the re-amplification of light chain from cDNA of mAb 9B9 and subsequent sub-cloning in phagemid vector pSex 81 using Mlul and Noil restriction sites were designed:

forward
(SEQ ID NO: 3)
5′-aattttcagaagcacgcgtagatatccaggctgttgtgact-3′
reverse
(SEQ ID NO: 4)
5′-gaagatggatccagcggccgcggctggcctaggaca-3′

Heavy and light chain sequences were sub-cloned into phagemid vector pSEX 81 and several rounds of selection were performed using human angiotensin-converting enzyme (ACE) absorbed on the plate for selection of immunoreactive phages. Bacterial clones infected with positively selected phages were screened by PCR reaction for presence of proper size PCR products (around 1000 bp) which consisted of full size scFv 9B9 and plasmid's sequences upstream and downstream of scFv 9B9 insert. Three PCR products with size approximately 1000 bp were purified for following sequence analysis. The obtained single chain was analyzed in two formats: (i) scFv expressed on the phage surface, and (ii) scFv as protein.

Selected phages as a population and single clones were tested for their specificity to human and rat ACE using (i) phage ELISA on plates covered with human and rat ACEs; (ii) using cells expressing human and rat ACE; and (iii) in vivo in the rats. Results were confirmed in the tests where scFv 9B9 was used as a protein (scFv 9B9 sequence was subcloned in to expression vector pOPE and scFv 9B9 was expressed in XL-1 blue E. coli as a soluble protein).

Nucleotide Sequence of the Heavy and the Light Chains of 9B9 scFv Linked by Nucleotide Linker:

Heavy chain (Vₕ)
(SEQ ID NO: 5)
cag gtg cag ctg aag gag tca gga cct ggc ctg gtg gcg
ccc tca cag agc ctg tcc atc act tgc act gtc tct ggg
ttt tca tta acc acc tat ggt gta cac tgg gtt cgc cag
cct cca gga aag ggt ctg gag tgg ctg gga gta ata tgg
ggt ggt gga aac aca aat tat aat tcg gct ctc atg tcc
aga ctg aac atc acc aaa gac aac tcc aag cgc caa gtt
ttc tta aaa atg aac agt ctg caa gct gat gac aca ggc
atg tac tac tgt gcc aga ggg tgg gac tcc tgg ggc caa
ggc acc act ctc act gtc tcc tca gcc aaa acg aca ccc
cca aag ctt
Linker:
(SEQ ID NO: 6)
gaa gaa ggt gaa ttt tca gaa gca cgc
Light chain (V_{L}):
(SEQ ID NO: 7)
gta cag gct gtt gtg act cag gaa tct gca ctc acc aca
tca cct ggt gaa aca gtc aca ctc act tgt cgc tca agt
act ggg gct gta aca act aat aac tat gcc aac tgg gtc
caa gaa aat cca gat cat tta ttc act ggt cta ata gat
ggt acc aac acc cga tct cca ggt gtt cct gcc aga ttc
tca ggc tcc ctg att gga gac aaggct gcc ctc acc atc
aca ggg gca cag act gag gat gag gca ata tat ttc tgt
gct cta tgg tac agt aac cat tgg gtg ttc ggt gga gga
acc aaa ctg act gtc cta ggc cag

Amino Acid Sequence of the Heavy and the Light Chains of 9B9 scFv Linked by Polypeptide Linker:

```
Heavy chain (Vₕ):
(SEQ ID NO: 8)
                                                          CDR-H1
QVQLKESGPGLVAPSQSLSITCTVSGFSLTTYGVHWVRQPPGKGLEWLG
    CDR-H2 Sug
VIWGGGNTNYNSALMSRLNITKDNSKRQVFLKMNSLQADDTGMYYCAR
CDR-H3
GWDSWGQGTTLTVSSAKTTPPKL
Linker
(SEQ ID NO: 9)
EEGEFSEAR
Light chain (V_{L}):
(SEQ ID NO: 10)
                                              CDR-L1
VQAVVTQESALTTSPGETVTLTCRSSTGAVTTNNYANWVQENPDHLFTG
        CDR-L2
LIDGTNTRSPGVPARFSGSLIGDKAALTITGAQTEDEAIYFC
CDR-L3
ALWYSNHWVFGGGTKLTVLGQ
```

### Example 13

Increased Production of scFv 9B9 Fragments in CHO Cell In Vitro

Antibody fragments are valuable tools for immunotherapy. However, the low production of antibody fragments and their derivatives in the culture of mammalian cells could be an obstacle for their evaluation in animal models and for clinical applications. It has been shown recently that low cultivation temperature of CHO cells increased the productivity of the recombinant protein (1) and anti-ERbB2 scFc-Fc-IL2 fusion (2).

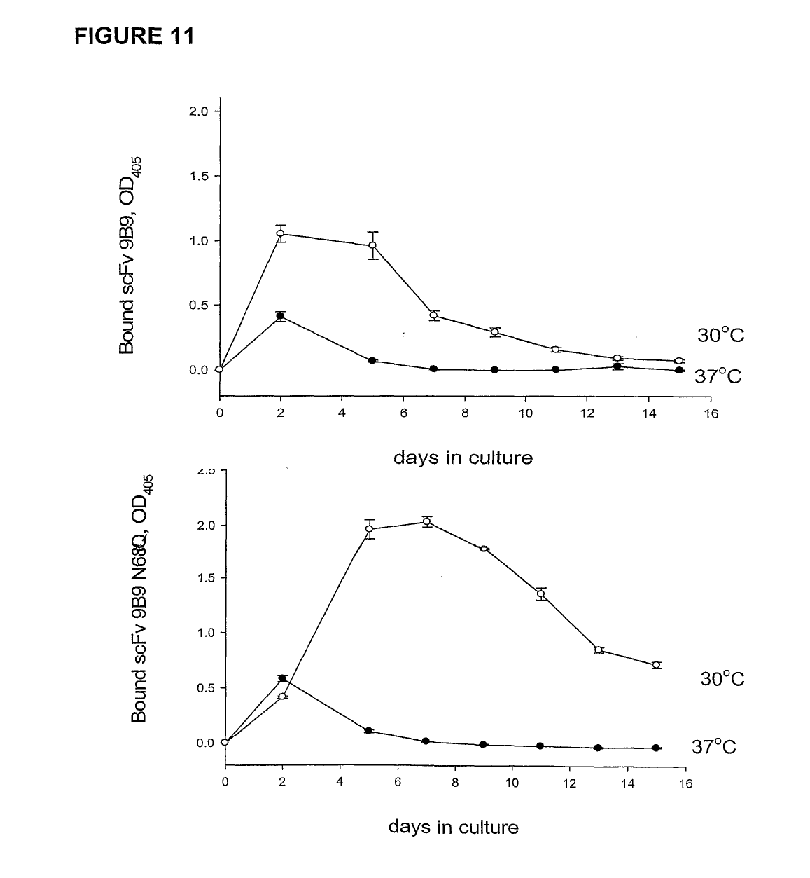

FIG. 11 shows data in which the cultivation of CHO cells at 30° C. increases the production of 9B9scFv and 9B9scFv N68Q by CHO cells transiently expressing these antibody fragments.

### Example 14

### Generation of Mouse 9B9scFv N68Q Human Fc Fragment Chimera

Generation of fusions of single-chain antibody with human Fc fragment represent one of the method to (i) increase the stability of scFv; (ii) increase the molecular weight of the fusion, and therefore to decrease quick removal scFv through the kidney, and as result—higher concentration in the blood and targeted organ in vivo, (iii) longer half life in vivo, (iv) increase of affinity due to dimeric nature, and (v) decreased immunogenicity due to human origin of Fc fragment for clinical applications. Currently several murine-human antibody fragments chimeras are in pre-clinical stage, and Abciximab (murine/human chimera) (Eli Lilly) is FDA approved (4).

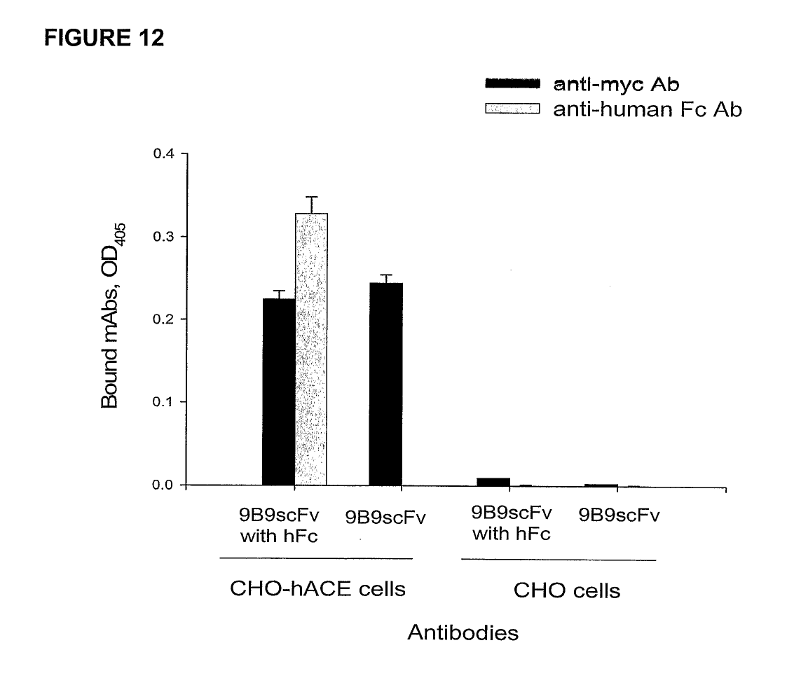

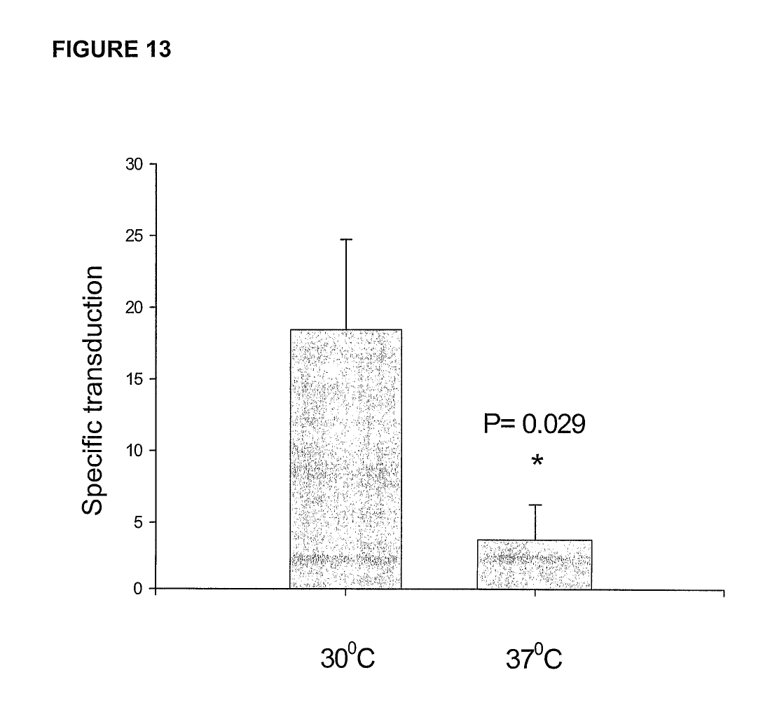

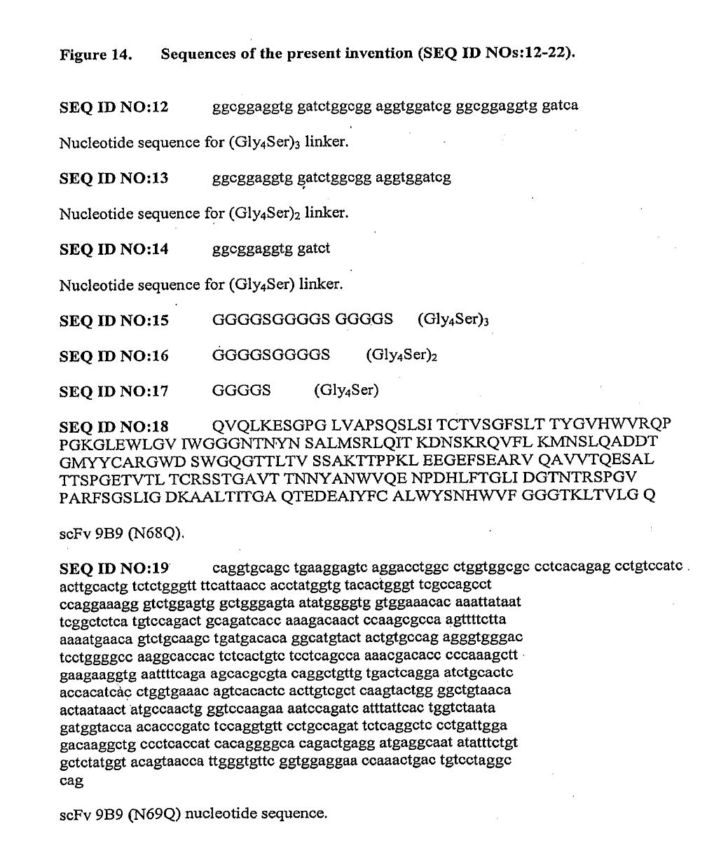

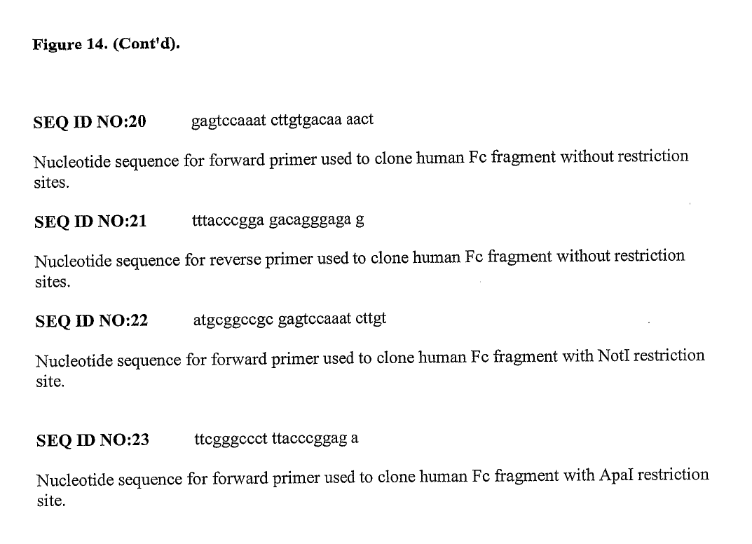

The production of functional fusion 9B9 scFv N68Q with human Fc in CHO cells in vitro is demonstrated here. FIG. 12 shows that 9B9scFv N68QhFc fusion specifically binds with human ACE and can be revealed by both anti-myc antibody and by anti-human Fc specific antibody, whereas 9B9 scFvN68Q without human Fc can be revealed only by anti-myc antibody.

### Example 15

### Viral Surface Expression of scFv 9B9

To date, a very few studies have attempted in vivo targeting and transfection of a gene of interest in the pulmonary endothelium. In our own studies, we have used a bi-specific Fab antibody fragment conjugate approach to deliver reporter or eNOS genes to the pulmonary circulation using adenovirus as vehicle. Such bi-specific mAbs are capable of simultaneously binding two different molecules at the same time: one arm binds to adenovirus, the other arm to ACE expressed on the surface of endothelial cells. However, preparation of such bi-specific antibodies requires their chemical conjugation and can be heterogeneous from lot to lot with respect to antigen-binding capacity.

An alternative approach is to target pulmonary endothelial cells through the smallest antibody fragments (e.g. single-chain fragments) against endothelial cell antigens genetically engineered in the viral envelope. The development of such a single-component targeted vector will simplify production and ensure homogeneity of vector production. Therefore, the expression of 9B9 scFv on the surface of viruses represent further advanced approach to deliver therapeutic genes to the pulmonary circulation. Retroviral envelope gene gp70 was modified with scFv 9B9 gene by incorporating the scFv 9B9 cDNA at +1 position of the gene encoding gp70. A plasmid encoding amphotropic gp70 contained cDNA for scFv 9B9 as revealed by restriction analysis (data not shown). The unique restriction site was introduced into +1 position of a plasmid encoding amphotropic envelope gene using site directed mutagenesis. In addition, the amino acid linker AAIEGR (SEQ ID NO:11) was introduced to the 3′ end of scFv to accommodate steric interaction between single chain and the domain of the chimeric envelope. Subsequently, modified 9B9 scFv was subcloned in the +1 position of the envelope gene. The final product was verified by restriction and sequence analysis.

### Example 16

### Transduction Efficiency

Chimeric retroviruses were tested in vitro for their ability to specifically transduce a cell line expressing human ACE, wherein the transduction occurs via 9B9scFv human ACE interactions. RLMVEC expressing human ACE was used as a model of pulmonary endothelium. RLMVEC and RLMVEC-hACE were infected with control or chimeric virus encoding Lac Z reporter gene. In 48 hours post-infection, cells were stained for X-gal activity. Number of X-gal positive cells (further will be referred as transductional units (TU)) was estimated for control and chimeric virus in each cell line. The efficiency to transduce (TE) RLMVEC-hACE was calculated as ratio of TU on RLMVEC-hACE/TU on RLMVEC for control and chimeric virus. FIG. 6 below demonstrates that TE of chimeric virus (e.g. the ability to transduce RLMVEC-hACE) several times exceeded that one for control virus. The specificity of transduction of RLMVEC-hACE by chimeric virus was estimated as ratio of TE of chimeric virus to TE of control virus.

As an example, we modified retroviral envelope gene gp70 with 9B9scFv gene by incorporating 9B9scFv cDNA at +1 position of gene encoding gp70. Plasmid encoding amphotropic gp70 contains cDNA for 9B9scFv as revealed by restriction analysis (data not shown).

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

1. An anti-ACE antibody which consists of SEQ ID NO:8 and SEQ ID NO:10, wherein SEQ ID NO:8 and SEQ ID NO:10 are linked via an amino acid linker.

2. An anti-ACE antibody encoded by the nucleic acid sequence denoted in SEQ ID NO: 1.

3. A method for targeting a selected therapeutic to an ACE-expressing tissue of an animal comprising administering to an animal a selected therapeutic conjugated to an anti-ACE single chain fragment antibody.

4. The method of claim 3, wherein the antibody comprises an amino acid sequence selected from the group consisting of SEQ ID NO:2 and SEQ ID NO:18.

5. The method of claim 3, wherein the antibody comprises SEQ ID NO:8 linked to SEQ ID NO: 10 via a flexible amino acid linker sequence selected from the group of SEQ ID NO:13, SEQ ID NO:14, and SEQ ID NO:15.

6. The antibody of claim 1, wherein the antibody is attached to a material selected from the group consisting of a radioisotope, a toxin, a plasminogen activator, a catalase, a superoxide dismutase, a cytotoxic agent and a detectable label.

7. The method of claim 6, wherein the plasminogen activator is selected from the group consisting of tissue-type PA (t-PA), urokinase PA (u-PA), and streptokinase.

8. The method of claim 3, wherein the antibody is an antibody multimer.

9. The method of claim 3, wherein the ACE-expressing tissue of the animal is lung tissue.

10. An isolated DNA molecule comprising a nucleotide sequence selected from the group consisting of SEQ ID NO:1, SEQ ID NO:5, SEQ ID NO:7, and SEQ ID NO:19.

11. A vector comprising the isolated DNA of claim 10.

12. An isolated host cell transformed with the vector of claim 11.

13. A method of producing an anti-ACE scFv antibody, the method comprising the steps of: (a) providing a cell comprising the isolated DNA of claim 2; and (b) culturing the cell under conditions that permit expression of the antibody from the isolated DNA, to thereby produce the antibody.

14. The host cell of claim 12, wherein the cell is selected from the group consisting of bacterial cells, mammalian cells, plant cells, and insect cells.

15. A method for radioimaging comprising administering to a subject an effective amount of a radiolabeled scFv 9B9 antibody complex.

16. The method of claim 18, wherein the complex is radiolabeled with an ion selected from the group consisting of iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

17. The method of claim 6, wherein the detectable label is selected from the group consisting of iodine (¹³¹I, ¹²⁵I, ¹²³I, ¹²¹I), carbon (¹⁴C), sulfur (³⁵S), tritium (³H), indium (^{115m}In, ^{113m}In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

18. A method for directing cells to ACE-expressing tissue of a mammal comprising:
(a) transfecting the cells with the vector of claim 14, and
(b) administering the cells to the mammal.

19. An anti-ACE antibody which consists of SEQ ID NO:24 and SEQ ID NO:10, wherein SEQ ID NO:24 and SEQ ID NO:10 are linked via an amino acid linker.

20. An anti-ACE antibody encoded by the nucleic acid sequence denoted in SEQ ID NO:19.

21. The antibody of claim 19, wherein the antibody is attached to a material selected from the group consisting of a radioisotope, a toxin, a plasminogen activator, a catalase, a superoxide dismutase, a cytotoxic agent and a detectable label.

22. The antibody of claim 19, wherein the linker is selected from the group of SEQ ID NO:13, SEQ ID NO:14, and SEQ ID NO:15.

23. The antibody of claim 19, wherein the antibody is fused to a human Fc antibody fragment.


SEQUENCE LISTING (25 sequences)

SEQ ID NO: 1 - DNA, 723 bp, Artificial Sequence
  Description of Artificial Sequence Synthetic polynucleotide
caggtgcagctgaaggagtcaggacctggcctggtggcgccctcacagagcctgtccatcacttgcactgtctctgggttttcattaaccacctatggtgtacactgggttcgccagcctccaggaaagggtctggagtggctgggagtaatatggggtggtggaaacacaaattataattcggctctcatgtccagactgaacatcaccaaagacaactccaagcgccaagttttcttaaaaatgaacagtctgcaagctgatgacacaggcatgtactactgtgccagagggtgggactcctggggccaaggcaccactctcactgtctcctcagccaaaacgacacccccaaagcttgaagaaggtgaattttcagaagcacgcgtacaggctgttgtgactcaggaatctgcactcaccacatcacctggtgaaacagtcacactcacttgtcgctcaagtactggggctgtaacaactaataactatgccaactgggtccaagaaaatccagatcatttattcactggtctaatagatggtaccaacacccgatctccaggtgttcctgccagattctcaggctccctgattggagacaaggctgccctcaccatcacaggggcacagactgaggatgaggcaatatatttctgtgctctatggtacagtaaccattgggtgttcggtggaggaaccaaactgactgtcctaggccag

SEQ ID NO: 2 - protein, 241 aa, Artificial Sequence
  Description of Artificial Sequence Synthetic polypeptide
QVQLKESGPGLVAPSQSLSITCTVSGFSLTTYGVHWVRQPPGKGLEWLGVIWGGGNTNYNSALMSRLNITKDNSKRQVFLKMNSLQADDTGMYYCARGWDSWGQGTTLTVSSAKTTPPKLEEGEFSEARVQAVVTQESALTTSPGETVTLTCRSSTGAVTTNNYANWVQENPDHLFTGLIDGTNTRSPGVPARFSGSLIGDKAALTITGAQTEDEAIYFCALWYSNHWVFGGGTKLTVLGQ

SEQ ID NO: 3 - DNA, 41 bp, Artificial Sequence
  Description of Artificial Sequence Synthetic primer
aattttcagaagcacgcgtagatatccaggctgttgtgact

SEQ ID NO: 4 - DNA, 36 bp, Artificial Sequence
  Description of Artificial Sequence Synthetic primer
gaagatggatccagcggccgcggctggcctaggaca

SEQ ID NO: 5 - DNA, 360 bp, Murinae gen. sp.
caggtgcagctgaaggagtcaggacctggcctggtggcgccctcacagagcctgtccatcacttgcactgtctctgggttttcattaaccacctatggtgtacactgggttcgccagcctccaggaaagggtctggagtggctgggagtaatatggggtggtggaaacacaaattataattcggctctcatgtccagactgaacatcaccaaagacaactccaagcgccaagttttcttaaaaatgaacagtctgcaagctgatgacacaggcatgtactactgtgccagagggtgggactcctggggccaaggcaccactctcactgtctcctcagccaaaacgacacccccaaagctt

SEQ ID NO: 6 - DNA, 27 bp, Artificial Sequence
  Description of Artificial Sequence Synthetic oligonucleotide
gaagaaggtgaattttcagaagcacgc

SEQ ID NO: 7 - DNA, 336 bp, Homo sapiens
gtacaggctgttgtgactcaggaatctgcactcaccacatcacctggtgaaacagtcacactcacttgtcgctcaagtactggggctgtaacaactaataactatgccaactgggtccaagaaaatccagatcatttattcactggtctaatagatggtaccaacacccgatctccaggtgttcctgccagattctcaggctccctgattggagacaaggctgccctcaccatcacaggggcacagactgaggatgaggcaatatatttctgtgctctatggtacagtaaccattgggtgttcggtggaggaaccaaactgactgtcctaggccag

SEQ ID NO: 8 - protein, 120 aa, Murinae gen. sp.
QVQLKESGPGLVAPSQSLSITCTVSGFSLTTYGVHWVRQPPGKGLEWLGVIWGGGNTNYNSALMSRLNITKDNSKRQVFLKMNSLQADDTGMYYCARGWDSWGQGTTLTVSSAKTTPPKL

SEQ ID NO: 9 - protein, 9 aa, Artificial Sequence
  Description of Artificial Sequence Synthetic peptide
EEGEFSEAR

SEQ ID NO: 10 - protein, 112 aa, Murinae gen. sp.
VQAVVTQESALTTSPGETVTLTCRSSTGAVTTNNYANWVQENPDHLFTGLIDGTNTRSPGVPARFSGSLIGDKAALTITGAQTEDEAIYFCALWYSNHWVFGGGTKLTVLGQ

SEQ ID NO: 11 - protein, 6 aa, Artificial Sequence
  Description of Artificial Sequence Synthetic peptide
AAIEGR

SEQ ID NO: 12 - DNA, 45 bp, Artificial Sequence
  Description of Artificial Sequence Synthetic oligonucleotide
ggcggaggtggatctggcggaggtggatcgggcggaggtggatca

SEQ ID NO: 13 - DNA, 30 bp, Artificial Sequence
  Description of Artificial Sequence Synthetic oligonucleotide
ggcggaggtggatctggcggaggtggatcg

SEQ ID NO: 14 - DNA, 15 bp, Artificial Sequence
  Description of Artificial Sequence Synthetic oligonucleotide
ggcggaggtggatct

SEQ ID NO: 15 - protein, 15 aa, Artificial Sequence
  Description of Artificial Sequence Synthetic peptide
GGGGSGGGGSGGGGS

SEQ ID NO: 16 - protein, 10 aa, Artificial Sequence
  Description of Artificial Sequence Synthetic peptide
GGGGSGGGGS

SEQ ID NO: 17 - protein, 5 aa, Artificial Sequence
  Description of Artificial Sequence Synthetic peptide
GGGGS

SEQ ID NO: 18 - protein, 241 aa, Artificial Sequence
  Description of Artificial Sequence Synthetic polypeptide
QVQLKESGPGLVAPSQSLSITCTVSGFSLTTYGVHWVRQPPGKGLEWLGVIWGGGNTNYNSALMSRLQITKDNSKRQVFLKMNSLQADDTGMYYCARGWDSWGQGTTLTVSSAKTTPPKLEEGEFSEARVQAVVTQESALTTSPGETVTLTCRSSTGAVTTNNYANWVQENPDHLFTGLIDGTNTRSPGVPARFSGSLIGDKAALTITGAQTEDEAIYFCALWYSNHWVFGGGTKLTVLGQ

SEQ ID NO: 19 - DNA, 723 bp, Artificial Sequence
  Description of Artificial Sequence Synthetic polynucleotide
caggtgcagctgaaggagtcaggacctggcctggtggcgccctcacagagcctgtccatcacttgcactgtctctgggttttcattaaccacctatggtgtacactgggttcgccagcctccaggaaagggtctggagtggctgggagtaatatggggtggtggaaacacaaattataattcggctctcatgtccagactgcagatcaccaaagacaactccaagcgccaagttttcttaaaaatgaacagtctgcaagctgatgacacaggcatgtactactgtgccagagggtgggactcctggggccaaggcaccactctcactgtctcctcagccaaaacgacacccccaaagcttgaagaaggtgaattttcagaagcacgcgtacaggctgttgtgactcaggaatctgcactcaccacatcacctggtgaaacagtcacactcacttgtcgctcaagtactggggctgtaacaactaataactatgccaactgggtccaagaaaatccagatcatttattcactggtctaatagatggtaccaacacccgatctccaggtgttcctgccagattctcaggctccctgattggagacaaggctgccctcaccatcacaggggcacagactgaggatgaggcaatatatttctgtgctctatggtacagtaaccattgggtgttcggtggaggaaccaaactgactgtcctaggccag

SEQ ID NO: 20 - DNA, 24 bp, Artificial Sequence
  Description of Artificial Sequence Synthetic primer
gagtccaaatcttgtgacaaaact

SEQ ID NO: 21 - DNA, 21 bp, Artificial Sequence
  Description of Artificial Sequence Synthetic primer
tttacccggagacagggagag

SEQ ID NO: 22 - DNA, 25 bp, Artificial Sequence
  Description of Artificial Sequence Synthetic primer
atgcggccgcgagtccaaatcttgt

SEQ ID NO: 23 - DNA, 21 bp, Artificial Sequence
  Description of Artificial Sequence Synthetic primer
ttcgggccctttacccggaga

SEQ ID NO: 24 - protein, 120 aa, Artificial Sequence
  Description of Artificial Sequence Synthetic polypeptide
QVQLKESGPGLVAPSQSLSITCTVSGFSLTTYGVHWVRQPPGKGLEWLGVIWGGGNTNYNSALMSRLQITKDNSKRQVFLKMNSLQADDTGMYYCARGWDSWGQGTTLTVSSAKTTPPKL

SEQ ID NO: 25 - protein, 6 aa, Artificial Sequence
  Description of Artificial Sequence Synthetic 6x His tag
HHHHHH